(19) United States
(12) Patent Application Publication
(10) Pub. No.: US 2004/0248234 A1
(43) Pub. Date: Dec. 9, 2004

(54) Sensitive and selective in vitro assay for the detection of reactive drug intermediates

(75) Inventors: Mark J. Cole, Mystic, CT (US); Shawn P. Harriman, Charlestown, RI (US); John R. Soglia, Westerly, RI (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/863,164

(22) Filed: Jun. 8, 2004

Related U.S. Application Data
(60) Provisional application No. 60/546,443, filed on Feb. 19, 2004.
(60) Provisional application No. 60/477,472, filed on Jun. 9, 2003.

(51) Int. Cl. C12Q 1/68; C12Q 1/26

(52) U.S. Cl. 435/25

(57) ABSTRACT

In vitro processes for detecting one or more reactive metabolites that may be formed from a substrate (e.g., a drug or a potential drug candidate) by an enzyme system is disclosed. The substrate is contacted in a mixture with an enzyme system (e.g., with a microsomal drug metabolizing enzyme system, such as a P450 system) to form reactive species (e.g., reactive metabolites), which in the same or a different mixture are contacted with a compound (e.g., glutathione ethyl ester) that reacts with the reactive species to form detectable species (e.g., glutathione ethyl ester conjugates). Preferably, solid phase extraction, high performance liquid chromatography, electrospray ionization, and tandem triple quadropole mass spectrometry are used for detection. The processes may be used in the early stages of a drug discovery program, as well as in other contexts.

### CROSS REFERENCE TO RELATED APPLICATIONS

This Patent application claims priority of U.S. Provisional patent application No. 60/477,472 filed Jun. 9, 2003 and U.S. Provisional patent application No. 60/546,443, filed Feb. 19, 2004.

### FIELD OF THE INVENTION

This invention provides in vitro methods for screening compounds (e.g., possible drug candidates) to determine if those compounds or substrates, in vivo, would likely produce reactive metabolites that might be deleterious. More specifically, this invention provides in vitro screening methods that contact the substrate(s) to be screened with an enzyme system in the presence of, e.g., glutathione ethyl ester, to determine if reactive metabolites (intermediates) of the substrate(s) are formed.

### BACKGROUND OF THE INVENTION

Combinatorial chemistry synthesis has revolutionized modern drug discovery by enabling rapid production of large numbers of compounds as possible drug candidates. As a result, high-throughput screening methods have been developed to quickly screen the large number of compounds that are synthesized. Assays that identify compounds having less desirable physical, chemical, and/or biological properties early in the screening process are invaluable in identifying compounds because undesirable compounds can be removed from further consideration as soon as possible. Hence, more of the available resources can be expended on promising compounds, thereby increasing the likelihood that new beneficial drugs will reach patients sooner.

Inadequate in vivo detoxification of reactive metabolites formed from drugs is believed to be a pathogenic mechanism for tissue necrosis, carcinogenicity, teratogenicity, and immune mediated toxicity. Development of effective assays for detecting species possibly implicated in toxicity that could be used early in the discovery program would be highly advantageous.

United States Patent Application Publication No. US 2002/0034729 discloses that electrophilic reactive metabolites formed in vivo (e.g., as a result of liver metabolism) manifest their toxicity by covalently binding to nucleophilic groups of vital cellular proteins and nucleic acids. The application describes a high-throughput method for identifying drug candidates that produce reactive metabolites. The drug candidate is incubated with a P450 system microsomal drug metabolizing enzyme system in the presence of glutathione. The enzyme system causes reactive metabolites to form from the drug candidate if the candidate is a precursor for such species. In the body, glutathione, which is metabolically stable and is naturally present in vivo (it is widely distributed in plant and animal cells), binds through its nucleophilic sulfhydryl group with the reactive electrophilic moieties of reactive species to form stable S-substituted conjugates (or adducts), thereby providing a natural mechanism for preventing such reactive species from binding with vital cellular constituents. In the incubation mixture, the glutathione binds with reactive metabolites formed by the P450 microsomal drug metabolizing enzyme system, and the resulting glutathione conjugates are detected using tandem mass spectrometry. The collision-induced dissociation that is part of the mass spectrometry procedure causes the glutathione conjugates to undergo the neutral loss of a pyroglutamic acid moiety (weighing 129 Dalton or Da). From that loss, one infers that glutathione conjugates were present in the test mixture.

In vitro assays that are highly specific for reactive species (e.g., reactive metabolites), and that are sufficiently sensitive to detect reactive species at (i.e., when they result from) initial substrate concentrations that are relatively low are desirable. Furthermore, the need remains for such an in vitro assay that is specific and sensitive over a large range of widely differing starting compounds or substrates (e.g., potential drug candidates). In other words, the need remains for such an assay that is specific, sensitive, and widely applicable. The need also remains for such an assay that will detect reactive species that do not form stable adducts with glutathione and/or those formed by enzyme systems other than the P450 system (i.e., for non-microsomal systems as well as microsomal systems other than the P450 system). Moreover, the need remains for such assays that are rapid. Finally, the need also remains for such assays that can be automated. These needs are increasing and becoming more urgent as combinatorial chemistry methods produce ever-increasing numbers of compounds. US 2002/0034729 and all other documents discussed or otherwise referenced herein are hereby incorporated herein in their entireties for all purposes.

### BRIEF SUMMARY OF THE INVENTION

An invention that satisfies those needs and provides substantial benefits that will be apparent to one skilled in the art has developed.

Broadly speaking, in one aspect the present invention provides in vitro methods for detecting one or more reactive species that may be formed from a substrate by an enzyme system, one such method comprising:

(a) contacting in vitro in a first mixture the substrate with a substrate metabolizing enzyme system whereby one or more reactive species may be formed, the concentration of the substrate in the first mixture being no greater than about 50 μM;

(b) contacting in vitro the one or more reactive species, if formed, with a compound that reacts with the one or more reactive species to form one or more detectable species; and

(c) detecting at least some of the one or more detectable species formed in step (b).

In another aspect, the present invention provide in vitro methods for detecting one or more reactive metabolites that may be formed from a drug by an enzyme system, one such method comprising:

(a) contacting in vitro in a first mixture the drug with a drug metabolizing enzyme system whereby one or more reactive metabolites may be formed, the concentration of the drug in the first mixture being no greater than about 50 μM;

(b) contacting in vitro the one or more reactive metabolites, if formed, with a compound that reacts with the one or more reactive metabolites to form one or more detectable species; and

(c) detecting at least some of the one or more detectable species formed in step (b).

In yet another aspect, the invention provides in vitro methods for detecting one or more reactive metabolites that may be formed from a potential drug candidate by an enzyme system, one such method comprising:

(a) contacting in vitro in a first mixture the potential drug candidate with a drug metabolizing enzyme system whereby one or more reactive metabolites may be formed, the concentration of the potential drug candidate in the first mixture being no greater than about 50 μM;

(b) contacting in vitro the one or more reactive metabolites, if formed, with a compound that reacts with the one or more reactive metabolites to form one or more detectable species; and

(c) detecting at least some of the one or more detectable species formed in step (b).

In still another aspect, the invention provides in vitro methods of assessing the possible toxicity of drugs or of potential drug candidates in vivo comprising carrying out the foregoing methods on a drug or a potential drug candidate and assessing the drug or potential drug candidate as possibly being toxic in vivo if the level of one or more detectable species or of one or more reactive metabolites or of both is or are above one or more predetermined levels.

In a still further aspect, the invention provides sensitive and selective methods of assessing the possible toxicity of potential drug candidates in vivo, one such method comprising:

(a) providing a plurality of potential drug candidates;

(b) carrying out the foregoing methods on one of the potential drug candidates;

(c) repeating step (b) with a different one of the potential drug candidates; and

(d) assessing each of the potential drug candidates so tested as possibly being toxic in vivo if the respective level of one or more detectable species or of one or more reactive metabolites is or are above one or more predetermined levels.

In a preferred embodiment the step of contacting the one or more reactive species (e.g., reactive metabolites) with a compound (e.g., one comprising glutathione) to form one or more detectable species occurs in the first mixture. In another preferred embodiment the presence of the one or more reactive species (e.g., reactive metabolites) is assessed based on the results of detecting at least some of the one or more detectable species. In yet another preferred embodiment the substrate metabolizing enzyme system is normally present in vivo. In still another preferred embodiment the substrate metabolizing enzyme system comprises a microsomal substrate metabolizing enzyme system (e.g., a microsomal drug metabolizing enzyme system, such as a P450 system). In still another preferred embodiment the compound comprises a compound normally present in vivo, for example, a compound that normally reacts with reactive species in vivo (e.g., one comprising glutathione). In yet another preferred embodiment the one or more detectable species comprise a glutathione ethyl ester conjugate, in which case detecting the detectable species preferably comprises detecting the glutathione ethyl ester conjugate by detecting a species resulting from the loss by the glutathione ethyl ester conjugate of a pyroglutamic acid moiety. In still another embodiment the step of contacting in vitro in the first mixture the potential drug candidate with a drug metabolizing enzyme system is conducted with a substrate (e.g., drug or potential drug candidate) concentration in the first mixture no greater than 10 μM, 1 μM, 0.1 μM, or even 0.01 μM. In yet a further preferred embodiment the step of detecting at least some of the one or more detectable species comprises passing them into a mass spectrometer, which may be a tandem mass spectrometer and which may also be a triple quadropole mass spectrometer. In still one more preferred embodiment the one or more detectable species are subjected to electrospray ionization prior to passing them into the mass spectrometer. In yet another preferred embodiment the one or more detectable species are subjected to high performance liquid chromatography prior to subjecting them to electrospray ionization. In still another preferred embodiment the one or more detectable species are passed through a microbore capillary for the electrospray ionization. In yet a further preferred embodiment the one or more detectable species are subjected to high performance liquid chromatography prior to passing them into the mass spectrometer. In yet another preferred embodiment the one or more detectable species are subjected to solid phase extraction prior to passing them into the mass spectrometer. In some preferred embodiments the one or more embodiments set forth herein above may be combined to give yet another preferred embodiment.

An application of the present invention is its use in a drug discovery program. In such an application, the methods of this invention will be applied to a large number of compounds, which will be available in only small quantities but which can be screened rapidly and automatically. Thus, microwell plates with many different potential drug candidates may be brought near the various equipment and moved by robotic arms and manipulated for further processing (e.g., using a Tecan RSP9000 Robotic Sample Processor, marketed by Tecan U.S., Inc. (Durham, N.C., United States, a subsidiary of Tecan Group AG, Mannedorf, Switzerland)).

Still other features and advantages of this invention will be apparent to those skilled in the art from this description and the appendent claims.

### BRIEF DESCRIPTION OF THE DRAWINGS

FIG. 1 is the structure of glutathione showing R groups where modifications can be made to generate close-in-analogs of glutathione, which may be used in the methods of the invention; the molecule depicted in FIG. 1 is glutathione when the three R groups are each hydrogen;

FIG. 2 is a simplified overview of the pathway of acetaminophen metabolism in vivo in a human being;

FIG. 3 shows the product ion spectrum of the [M+H]⁺ ion of s-p-NBGSH (s-(p-nitrobenzyl)-glutathione sulfhydryl) (m/z 443 (mass/charge ratio of 443)), where fragmentation of the [M+H]⁺ ion was achieved using a collision offset voltage of −16V and a collision cell pressure of 1.0 mtorr (millitorr);

FIG. 4 shows a product ion scan of a microsomal incubation sample dosed with acetaminophen at 100 μM; fragmentation of the [M+H]⁺ ion (m/z 457) was achieved using a collision offset voltage of −25V and a collision cell pressure of 1.0 mtorr; trace A represents the total ion chromatogram (TIC) resulting from product ion analysis of the acetaminophen-dosed microsome sample; spectrum B represents the prominent product ions of [M+H]⁺; and spectrum C shows an enhanced view of the product ions in the 320 to 480 Da mass range of spectrum B;

FIG. 5 shows HPLC/SRM (high performance liquid chromatography/selected reaction monitoring) analysis of the four substrates that gave a positive response in the assay out of the twelve substrates in the assay substrate test set using glutathione as the compound in the assay, with substrate concentration at 10 μM using microsomal incubation samples;

FIG. 6 shows the product ion spectrum of the [M+H]⁺ ion of s-p-NBGSH-EE (s-(p-nitrobenzyl)-glutathione ethyl ester) (m/z 471 (mass/charge ratio of 471)), where fragmentation of the [M+H]⁺ ion was achieved using a collision offset voltage of −16V and a collision cell pressure of 1.0 mtorr (millitorr);

FIG. 7 shows the resulting peak areas plotted versus concentration when an standard curve for s-p-NBGSH and an standard curve for s-p-NBGSH-EE are prepared and subjected to selected reaction monitoring (SRM) analysis;

FIG. 8 shows the HPLC/TIC (high performance liquid chromatography/total ion chromatogram) analysis of the standards s-p-NBGSH and s-p-NBGSH-EE;

FIG. 9 shows the HPLC/product ion scan (high performance liquid chromatography/product ion scan) analysis of microsomal incubation samples dosed with clozapine at 250 μM;

FIG. 10 shows the results of analysis using the substrates that gave a positive response in the assay out of the twelve substrates in the assay substrate test set using GSH (acetaminophen, clozapine, rosiglitazone) by comparing the selected reaction monitoring (SRM) analysis for each substrate using glutathione, or alternatively, glutathione ethyl ester in microsomal assay;

FIG. 11 shows HPLC/SRM (high performance liquid chromatography/selected reaction monitoring) analysis of twelve substrates in the assay substrate test set using glutathione ethyl ester as the compound in the microsomal assay, with substrate concentration at 10 μM;

FIG. 12 shows the results of HPLC/SRM (high performance liquid chromatography/selected reaction monitoring) analysis of 50 substrates in the microsomal assay using glutathione ethyl ester as the compound, (10 μM substrate concentration) indicating whether each substrate was used as a positive control or negative control based on its activity in a human hepatotoxicity assay;

FIG. 13 shows the effect of P450 concentration on acetaminophen reactive metabolite (quinone-imine) formation (see FIG. 2); acetaminophen-GSH peak data summary utilizing 1, 0.5, and 0.25 μM P450 enzyme concentrations in the microsomal assay (10 μM acetaminophen concentration), which data were acquired using selected reaction monitoring.

These drawings are for illustrative purposes only and should not be used to unduly limit the scope of the present invention.

### DETAILED DESCRIPTION OF THE INVENTION

The methods of this invention are sensitive, selective, and widely applicable in vitro methods for detecting one or more reactive species that may be formed from a substrate by an enzyme system. The substrate may be any substance that allows the benefits of this invention to be achieved, e.g., a drug, a potential drug candidate, or still another type of compound.

For convenience, certain terms employed in the specification, examples, and appended claims are collected here. Unless defined otherwise, all technical and scientific terms used herein have the same meaning as commonly understood by one of ordinary skill in the art to which this invention belongs.

Abbreviations

The abbreviations used herein have their usual meaning in the art. However, to even further clarify the present invention, for convenience, the meaning of certain abbreviations are provided as follows: “CID” means collision induced dissociation; “CNL” means constant neutral loss; “Da” means Dalton; “ESI” means electrospray ioization; “GSH” means glutathione sulfhydryl; “s-p-GSH-EE” means s-p-glutathione sulfhydryl ethyl ester; “s-p-NBGSH” means s-p-nitrobenzyl glutathione sulfhydryl; “ml” means milliliter; “HPLC” means high performance liquid chromatography; “LOD” means level of detection; “LOQ” means level of quantitation; “μl” means microliter; “μM” means micromolar; “μM” means femtomolar; “mtorr” means millitorr; “MRM” means multiple reaction monitoring; “m/z” means mass to charge ratio; “P450” means cytochrome P 450 enzyme; “SRM” means selected reaction monitoring; “TIC” means total ion chromatogram; “SPE” means solid phase extraction; “U/ml” means units per microliter; “kV” means kilovolt; and “V” means volt.

The methods of this invention are sensitive and selective and are widely applicable. As further explained below, by “sensitive” is meant that the method can detect the analyte of interest at very low concentrations, by “selective” (or specific) is meant that the method can detect the analyte of interest with enough specificity to provide sufficient assurance that the analyte of interest is in fact being detected even in the presence of other substances (i.e., the rate of false positives and the rate of false negatives is low), and by “widely applicable” is meant that it can satisfactorily test a wide variety of different compounds. Thus, the assays of this invention are highly specific for reactive species (e.g., reactive metabolites), are sufficiently sensitive to detect reactive species at (i.e., when they result from) initial substrate concentrations of 10 μM or even far less, and are specific and sensitive over a large range of widely differing substrates (i.e., starting compounds such as potential drug candidates). The assays of this invention can detect reactive species that form stable adducts with glutathione ethyl ester as well as with other compounds (e.g., bovine pancreatic ribonuclease A, keyhole limpet hemocyanin, and others). The assays of this invention can detect reactive species formed by non-microsomal enzyme systems as well as microsomal enzyme systems (e.g., the P450 system).

The assays of the invention can be conducted rapidly and can be automated, both of which are advantageous in a drug discovery program, in which large numbers of potential drug candidates are screened in a short period of time for identifying the possibility of in vivo toxicity. By “automatic,” “automatically,” “automated,” and the like are meant that the various steps of the process of interest can be carried out substantially without operator intervention. Thus, for example, a sample holding means (e.g., a microwell plate) can contain dozens of testable mixtures to be subjected to various analytical methods (e.g., high performance liquid chromatography, mass spectrometry), with one potential drug candidate per microwell (in some cases in replicate) and some microwells possibly containing standards/controls. Each testable mixture can automatically be removed (e.g., aspirated) and automatically be tested to yield the desired analytical information (e.g., presence or concentration or amount of one or more species being used as markers). In some cases, desirably, the substrates to be assayed (e.g., potential drug candidates) can be automatically removed from their holders (e.g., sample cuvettes) and placed into a sample holding means (e.g., microwell plate) for contact with the one or more enzyme systems being utilized to form reactive species, followed by automatically contacting the reactive species (if any) with a compound that reacts with them (e.g., glutathione), followed by automatic recovery of the detectable species (e.g., by solid phase extraction), followed by the automated analytical steps.

The processes' being “automatic” or “automated” may also include an operator's being able to place the microwell plates (or other sample holding means) in a feeder and then having the associated apparatus automatically sequentially and repetitively place them in position for further processing (e.g., addition of the enzyme system, addition of a reactive compound, removal from the microwell for high performance liquid chromatography). Thus, a first microwell plate could automatically be moved into position and each of the microwells automatically processed and then, after all the microwell wells on that plate had been automatically processed, the plate could automatically be moved out of position and the next microwell plate could automatically be taken from the feeder and be automatically put into position and the process repeated until all microwells of all plates had been automatically processed.

One use of the methods of this invention is to determine or assess (with or without the help of a computer and with or without the help of mathematical methods, e.g., statistical techniques) the likelihood that a substrate, if introduced into an organism (e.g., from the plant or animal kingdom, e.g., a mammal, e.g., a human), will be acted upon in such a way, by in vivo metabolic processes, so as to produce one or more reactive species. “Reactive species” are potentially harmful species that are formed in vitro or in vivo by enzyme systems acting on a substrate and when produced by enzyme systems normally found in vivo, will often be referred to as “reactive metabolites.” Those species are potentially harmful because they may bind to vital molecular constituents of the organism and disrupt normal organic functions.

The P450 enzyme systems of the body (e.g., found in liver cells) help “detoxify” the human body. Thus, when a substrate, such as a drug, is introduced into the body, a P450 enzyme system may attempt to metabolize the drug. Unfortunately, a by-product of that process may be the production of reactive species. Some drugs, e.g., aspirin, have not been reported to produce such reactive species, while others, e.g., acetaminophen, have been reported to produce such species. In the body, glutathione, which is metabolically stable and is naturally present in vivo (it is widely distributed in plant and animal cells), binds through its nucleophilic sulfhydryl group with the reactive electrophilic moieties of reactive species to form stable S-substituted conjugates, which are excreted, thereby providing a natural mechanism for preventing such reactive species from binding with vital cellular constituents.

The contacting step of the present invention contacts the substrate (e.g., drug or potential drug candidate) with an enzyme system. One or more reactive species may be formed in this step. It is also possible that reactive species are not formed for a given substrate in the first contacting step. Any reactive species (reactive metabolites) formed in that step are then contacted with a compound that can react with those reactive species to form detectable species. If reactive species are not formed in the first contacting step, then contacting the first mixture with the compound in the second contacting step will not result in detectable species. This second contacting step preferably occurs (but need not occur) in the same mixture in which the first contacting step occurs. Thus, preferably, the substrate, enzyme system, and compound are contacted together in a single mixture so that any reactive species formed can come into contact directly with the compound. It should be understood that the compound need not meet the strict chemical definition of “compound.” The conditions under which the contacting steps occur are not critical and may be any conditions deemed appropriate by those skilled in the art. Headings are for illustrative purposes only and should not be used to unduly limit the scope of the present invention.

The Enzyme System.

The enzyme system used can be any enzyme system that allows the benefits of this invention to be achieved and need not be a microsomal enzyme system (e.g., a P450 microsomal enzyme system). The enzyme system used can be naturally occurring or man-made (synthetic). The enzyme system need not contain all of the compounds normally found in an organism and may omit some (if that is possible) and/or may use synthetic or simplified forms of some or all of the constituents of the system. The enzyme system used need not be one normally found in the organism of interest. An enzyme system “normally present,” “normally found,” and the like is an enzyme system that is endogenous to the organism in question, that is, an enzyme system originating in that organism. For example, if the possible toxicity of a substrate in a first organism is to be assessed using the method of this invention, the enzyme system used to produce reactive species for the in vitro method of this invention need not be an enzyme system normally found in that organism. In other words, in some cases it may be preferable (e.g., for economic or other reasons) to use a simplified enzyme system derived from non-human animals to assess the potential toxicity of drugs in humans. For assessing the possible toxicity of drugs or potential drug candidates in humans, a P450 enzyme system is preferred. See, e.g., U.S. Pat. Nos. 5,478,723; 5,891,696; and 6,004,927; which describe various enzyme systems.

The Compound

The compound that reacts with the reactive species (if they are formed) may be any compound that allows the benefits of this invention to be achieved. Where a P450 enzyme system is used, a substance comprising glutathione is the one such compound for contacting with the reactive species. Glutathione may also be referred to as γ-L-glutamyl-L-cysteinylglycine. It is a ubiquitous tri-peptide, particularly in mammals, and present in cells at high concentrations. Because of the nucleophilicity of its cysteinyl thiol group, glutathione functions as a co-factor in a number of biochemical reactions, and most importantly, glutathione is also involved in the detoxification of electrophilic and chemically reactive species arising during the biotransformation of xenobiotics. Thus, glutathione and chemically reactive species form glutathione conjugates which are detectable under some conditions. Such conditions may include: the conditions set forth describing the assays of the present patent application, conditions using radiolabelled assay constituents followed by the radioactive detection of conjugates, and conditions using derivitization techniques whereby conjugates are derivatized and subsequently detected. The known specificity and selectivity with which glutathione conjugates electrophilic drug metabolites is exploited by this invention to “trap” reactive metabolites.

Where a P450 enzyme system is used, a different compound that reacts with reactive species (if they are formed) comprises glutathione ethyl ester, which is what is known as a close-in-analog of glutathione and which is a preferred compound for contacting with the reactive species. Like glutathione, glutathione ethyl ester has a nucleophilic cysteinyl thiol group, so that glutathione ethyl ester and chemically reactive species form glutathione ethyl ester conjugates that are detectable under some conditions. Such conditions may include: the conditions set forth describing the assays of the present patent application, conditions using radiolabelled assay constituents followed by the radioactive detection of conjugates, and conditions using derivitization techniques whereby conjugates are derivatized and subsequently detected. Glutathione ethyl ester conjugates electrophilic drug metabolites and is specific, sensitive, and preferably exploited by this invention to “trap” reactive metabolites.

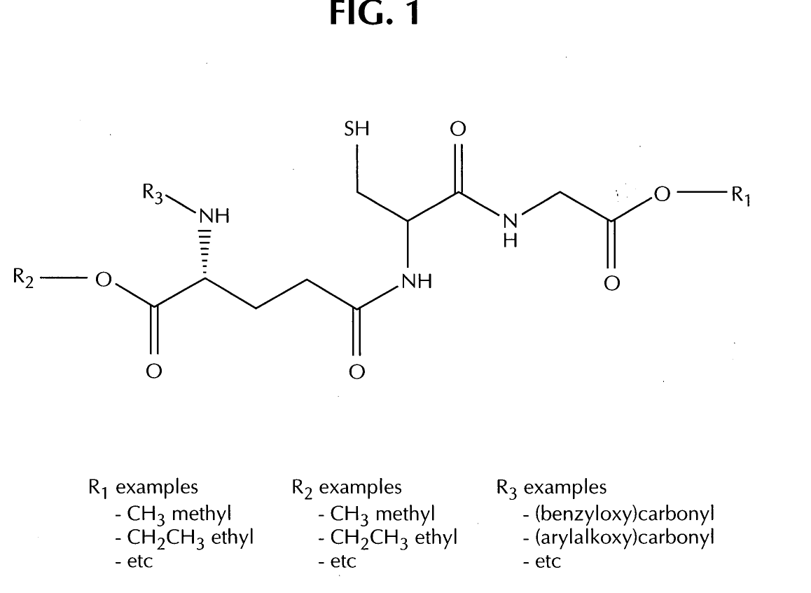

Compounds other than glutathione and glutathione ethyl ester may be used in the methods of the invention. For instance, other close-in-analogs of glutathione such as glutathione N-[(benzoyloxy)-carbonyl]methyl ester, and S-(N-methylcarbamoyl) glutathione may be used as the compound in the methods of the invention. Illustrated in FIG. 1 is the structure of glutathione. Positioned at the R₁, R₂, and R₃ sites in the glutathione molecule are hydrogen atoms. For purposes of the methods of the methods of the invention, the R₁ and R₃ sites represent the most probable sites of modification of glutathione. In FIG. 1, below the molecule are examples of substituents. Use of close-in-analogs of glutathione to detect reactive species is discussed in “Screening Strategy for the Detection of Derivatized Glutathione Conjugates by Tandem Mass Spectrometry”, Pearson et al., Anal. Chem., volume 62, pages 1827-1836 (1990). In the close-in-analog of glutathione which is preferred, glutathione ethyl ester, the R₁ group is an ethyl substituent, while R₂ and R₃ are hydrogen atoms. In selecting substituents in the design of close-in-analogs of glutathione, it is desirable to reduce hydrophobicity of the glutathione molecule, thus, use of glutathione ethyl ester will be discussed below.

However, not all reactive species react with thiol groups (which is the link through which glutathione and glutathione ethyl ester react with the reactive species), and such species may instead react with other nucleophilic side chains such as those belonging to lysine, histidine, aspartate, and glutamate, which are also present in vivo. In those cases, the use of a well-characterized protein (e.g., bovine pancreatic ribonuclease A, keyhole limpet hemocyanin), which contains a representative array of amino acids, may be useful in the present invention as the compound that binds with the reactive species.

A key feature of the methods of this invention is the relatively low concentration of substrate (e.g., drug or potential drug candidate) that can be used. Often, the concentration of a drug in vivo in body fluids (e.g., blood) is low (e.g., far less than 500 μM) and the concentrations of any reactive metabolites are typically significantly below the drug concentration. The assay of this invention has been tested using acetaminophen as the substrate, P450 as the enzyme system (at, e.g., a concentration of 0.25 μM), and glutathione as the compound that reacts with the reactive species to form the detectable species. When operating the mass spectrometer in CNL (constant neutral loss) mode to detect the glutathione conjugates (acetaminophen-GSH (glutathione sulfhydryl) conjugates), detection occurred with a substrate (acetaminophen) concentration of as little as 10 μM. When operating in SRM (selected reaction monitoring) mode, detection occurred with an acetaminophen substrate concentration of at little as 1 μM. For reasons set forth below, substrate concentrations far lower can be used for substrates of interest such as potential drug candidates (e.g., substrate concentrations no greater than 0.1 μM or even no greater than 0.01 μM).

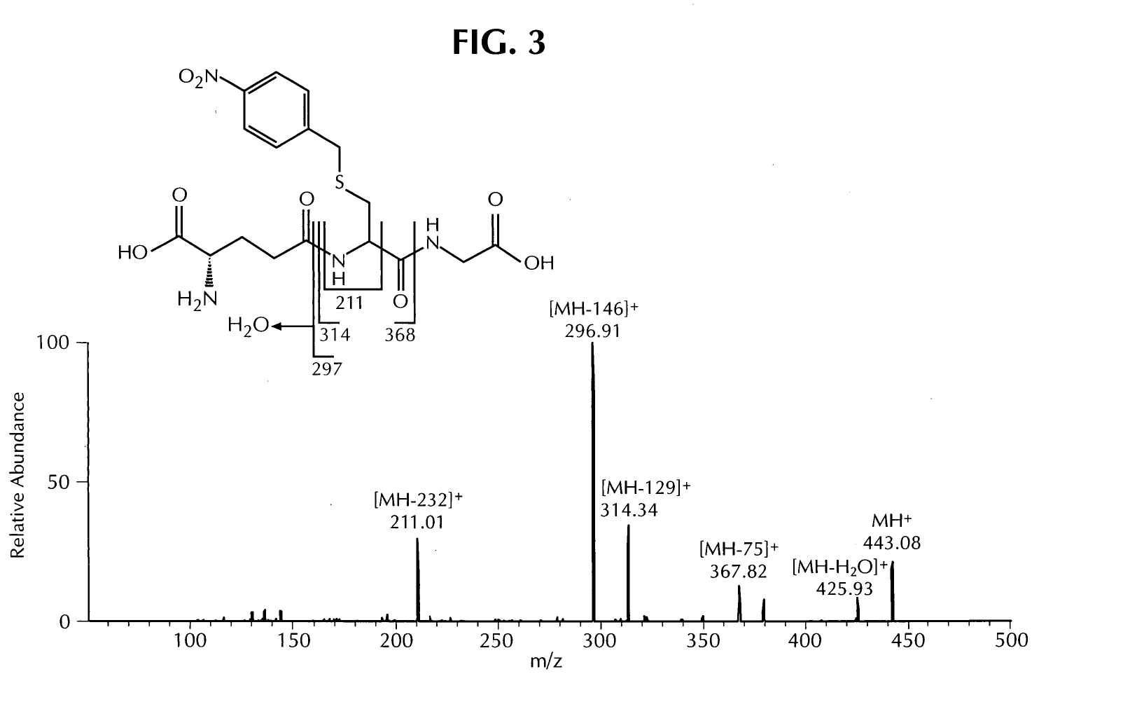

The factual underpinnings for this statement are as follows. Not wishing to be held to any particular theory, first, acetaminophen-GSH conjugates are very hydrophilic and only moderately retained in the sorbent from which the conjugates are recovered by SPE (solid phase extraction) for further processing. That results in poor recoveries during solid phase extraction (in other words, there is less conjugate to later be detected by the analytical equipment). Secondly, to achieve any retention/focusing of acetaminophen-GSH conjugates at the beginning of a capillary liquid chromatography column (to achieve a sharp peak), the starting mobile phase composition requires a high percentage of water. The use of high aqueous conditions during ESI (electrospray ionization) with a microbore column, which is preferably used between the high performance liquid chromatography and the mass spectrometry, is known to reduce analyte ionization efficiency. Those of ordinary skill in the art would recognize that the vast majority of the compounds to be screened as potential drug candidates will be more hydrophobic than acetaminophen, it is expected that the LOD (level of detection), as well as the LOQ (level of quantitation), would be far less than the 10 μM LOD in CNL mode or 1 μM LOD in SRM mode. Furthermore, using selected reaction monitoring (SRM) the methods of this invention enabled the detection (3 times signal-to-noise level) of 1.5 femtomole (fM) of s-p-NBGSH (on-column) and quantification (10 times signal-to-noise level) of 5.0 fM s-p-NBGSH (on-column) in. (A femtomole is 1×10⁻¹⁵ moles; s-p-NBGSH is s-(p-nitrobenzyl)-glutathione sulfhydryl, which is shown in the FIG. 3 product ion spectrum.)

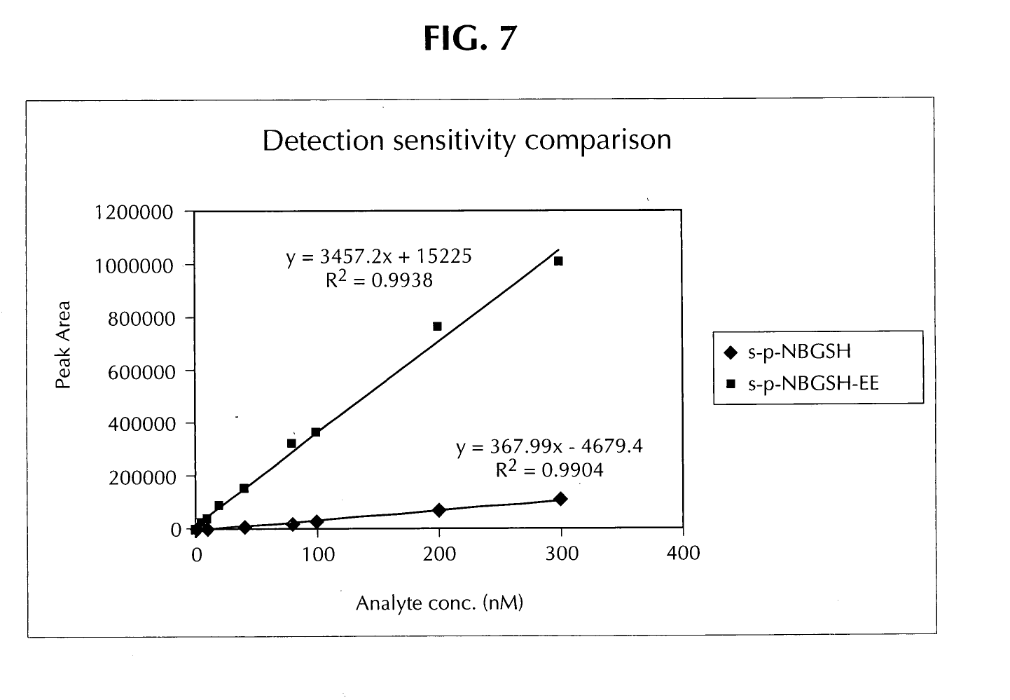

The assays of this invention may also be performed using glutathione ethyl ester as the compound that reacts with the reactive species to form the detectable species. A standard curve of serial dilutions of s-p-NBGSH and a standard curve of serial dilutions of s-p-NBGSH-EE were each prepared. The resulting peak areas for both s-p-NBGSH and s-p-NBGSH-EE were plotted versus concentration and the results are shown in FIG. 7. Least squares regression was used to generate the best-fit line and mathematical equation (y=m×+b) adjacent to each curve. Evident in the equations is an approximate 10-fold increase in slope that equates with a 10-fold increase in sensitivity for s-p-NBGSH-EE versus s-p-NBGSH. Thus, detection of s-p-NBGSH-EE is 10-fold more sensitive than detection of s-p-NBGSH using selected reaction monitoring (SRM) analysis. Not only is the detection of the standard, and s-p-NBGSH-EE 10 times more sensitive in SRM than the detection of s-p-GSH, but in the course of performing the experiments disclosed in the Examples set forth herein, it was found that detection of GSH-EE conjugates is more sensitive than the detection of GSH conjugates using mass spectrometry. Thus, the preferred compound for use in the methods of the invention is glutathione ethyl ester.

As can be seen from the description of the sensitivity with regard to glutathione and glutathione ethyl ester as just discussed, glutathione ethyl ester is about ten-fold more sensitive than glutathione in the methods of the invention. Again, not wishing to be held to any particular theory, this increased sensitivity is due, at least in part, to the increased hydrophobicity of the glutathione ethyl ester molecule in comparison to the glutathione molecule. This increased hydrophobicity arises, in part, because glutathione has two carboxylic acid groups, which exist because of the hydrogen molecules at R₁ and R₃ and glutathione ethyl ester has only one carboylic acid group, at R₃ (FIG. 1). This reduction in acid groups on the molecule may optimize the use of the mass spectrometer in positive ion mode, which results in more sensitive detection of glutathione ethyl ester conjugates. The use of glutathione ethyl ester, and conjugates which arise in its reaction with reactive species, during electrospray ionization with a microbore column, (which is preferably used between the high performance liquid chromatography and the mass spectrometry), increases the ionization characteristics of the compound, and its attendant conjugates, rendering mass spectrometry more sensitive.

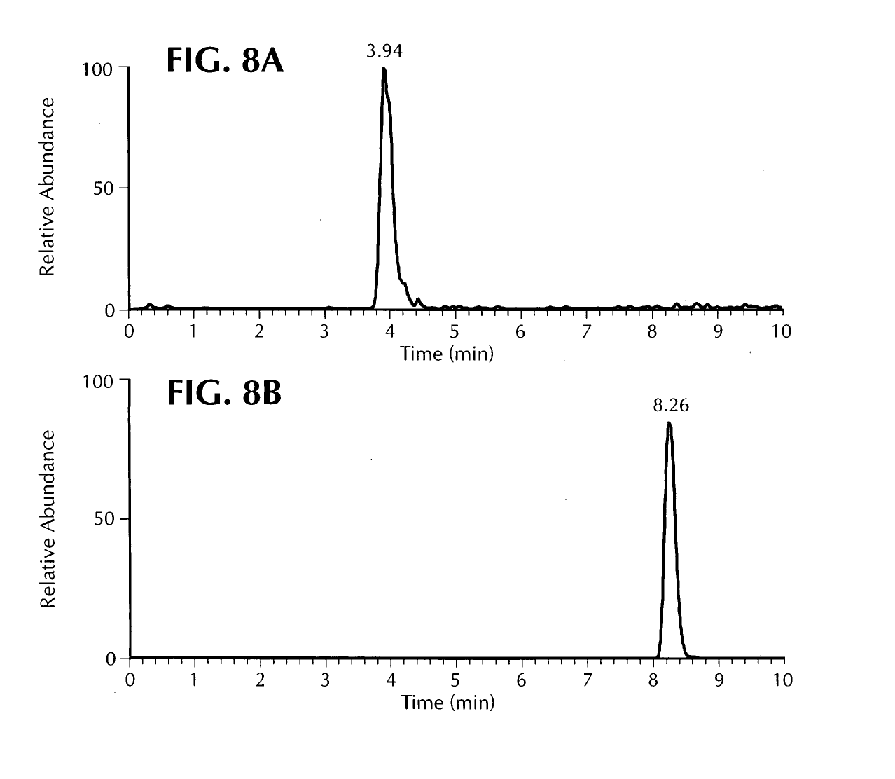

Another result of the increased hydrophobicity of the glutathione ethyl ester molecule, in relation to the glutathione molecule, is that the peaks in the chromatograms which result when the compounds, and/or the resulting conjugates, are subjected to high performance liquid chromatography, elute in a different location, giving rise to a chromatogram which is “cleaner” than that of glutathione and/or glutathione conjugates. As can be seen in FIG. 8, the peak containing s-p-nitrobenzyl-glutathione migrates at 3.94 minutes on Trace A, while the peak representing s-(p-nitrobenzyl)-glutathione ethyl ester migrates at 8.26 minutes on Trace B. The later elution of s-p-nitrobenzyl-glutathione ethyl ester occurs in an area of the trace where, in a chromatogram of a biological fluid, (e.g., in this case, microsomal incubation sample) little else is eluting, so such a peak is more readily detected.

The detection of reactive intermediates poses a challenge analytically because they are non-volatile, thermally labile, in some instances chemically unstable, and most lack a suitable chromaphore for ultraviolet detection. Thus, detecting such species can be technically challenging. However, it has been reported in the literature that glutathione can be used to stabilize such reactive intermediates by the formation of glutathione-reactive intermediate conjugates, which are, to some extent, stable and subject to characterization. However, for all the reasons discussed above, the use of glutathione ethyl ester has advantages over the use of glutathione in methods to detect reactive intermediates. Thus, the methods of the invention use glutathione ethyl ester, and other close-in-analogs of glutathione, to allow the detection of reactive intermediates.

Furthermore, as noted above, reactive intermediates are generally produced in very small quantities. Therefore, using glutathione ethyl ester as the compound to react with the reactive metabolites results in quite low levels of glutathione ethyl ester conjugates in any in vitro assay. To avoid this problem, prior reports have described using >100 μM substrate concentrations to produce reactive metabolite levels that could be detected using the reported analytical methodology. However, the problem in using such high substrate concentrations is that many of the compounds being synthesized for screening as potential drug candidates may not be soluble in an in vitro assay matrix at those high concentrations. This is a reason for the desirability of using low substrate concentrations in this type of drug discovery toxicity screen. To reduce the possibility of solubility problems arising with the wide range of potential drug candidates expected to be synthesized and screened, the analytical methods of this invention can routinely detect glutathione ethyl ester conjugates using a substrate concentration of ≦10 μM, resulting in only trace amounts of glutathione ethyl ester conjugates being formed and, therefore, being present to be detected. For all of these reasons, the analysis of glutathione ethyl ester conjugates in drug discovery requires a sensitive, selective, and widely applicable method that can handle a high sample throughput, which is what the present invention provides.

Reactive Species Detection

The processes of this invention preferably utilize a combination of solid phase extraction, high performance liquid chromatography, and mass spectrometry to detect the detectable species, e.g., the glutathione ethyl ester conjugates. Preferably, a triple quadropole mass spectrometer is used. In that device, collision induced dissociation (CID) causes the neutral loss of a pyroglutamic acid moiety (weighing 129 Da) when glutathione ethyl ester is the compound that reacts with the reactive species to form detectable species. From that loss, one infers that glutathione ethyl ester conjugates were present in the test mixture. From the presence of the glutathione ethyl ester conjugates, one then infers that reactive species were produced by the action of the enzyme system on the substrate. As indicated below, other moieties result from the collision induced dissociation of glutathione ethyl ester conjugates, but detection of the 129 Da moiety (i.e., the pyroglutamic acid moiety) is preferred.

Likewise, in the mass spectrometer, collision induced dissociation (CID) causes the neutral loss of a pyroglutamic acid moiety (weighing 129 Da) where glutathione is the compound that reacts with the reactive species to form detectable species. From that loss, one infers that glutathione conjugates were present in the test mixture. From the presence of the glutathione conjugates, one then infers that reactive species were produced by the action of the enzyme system on the substrate. As indicated below, while other moieties can result from the collision induced dissociation of glutathione conjugates, detection of a 129 Da moiety (i.e., the pyroglutamic acid moiety) is preferred.

Detecting at least some of the one or more detectable species formed during the in vitro contact of the reactive species with a compound that reacts with them (e.g., glutathione ethyl ester) includes detecting the detectable species directly as well as detecting them indirectly, such as by inferring their presence from the detection of a moiety characteristic of their presence, e.g., when they are further processed (for example, by the collision induced dissociation that is produced in the triple quadropole mass spectrometer). Use of other enzyme systems and/or compounds that react with any reactive species produced and/or other analytical equipment may or may not detect the presence of the detectable species directly.

### EXAMPLES

Various experiments using acetaminophen and s-(p-nitrobenzyl)-glutathione, as well as experiments using clozapine and s-(p-nitrobenzyl)-glutathione ethyl ester were run to illustrate the efficacy, specificity, and sensitivity of the method of this invention. In addition, an assay substrate test set was employed to compare the use of glutathione ethyl ester to glutathione in the methods of the invention. Thus, the results set forth herein demonstrate the invention works for its intended purpose.

FIG. 1

FIG. 1 is a representation of the structure of glutathione with three R groups R₁, R₂, and R₃, at which substitution can occur to generate close-in-analogs of glutathione. Where the substituents at R₁, R₂, and R₃ are hydrogen atoms, the molecule depicted in FIG. 1 is glutathione. The R₁ and R₃ sites are the most probable sites of modification of glutathione for purposes of the methods of the invention. Below the molecule are examples of substituents. For instance, glutathione ethyl ester has an ethyl substituent at R₁. The benzyloxy-carbonyl and arylalkoxy-carbonyl substituents are taken from the literature.

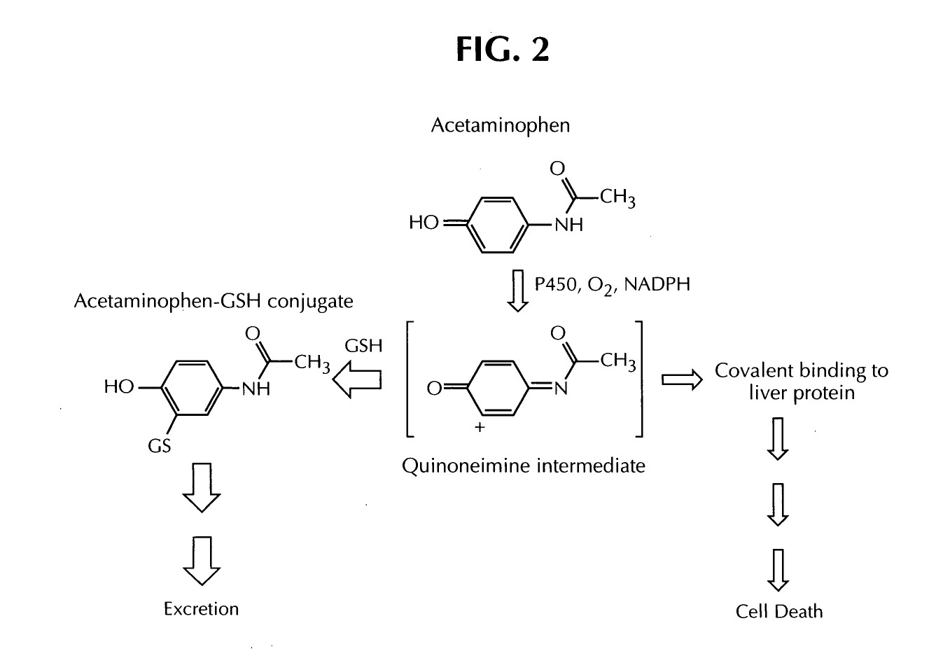

FIG. 2

FIG. 2 is a simplified overview of the pathway of acetaminophen metabolism in vivo in a human being. Acetaminophen is transformed into a quinone-imine intermediate (reactive metabolite) by the P450 enzyme system with oxygen and NADPH. That reactive metabolite (intermediate) can bind to liver proteins, causing cell death, or glutathione can bind to the reactive metabolite to form an acetaminophen-GSH conjugate, which is then excreted. Formation of the conjugate prevents cell death resulting from the detoxification of the drug by the liver (it can be such detoxification that results in the production of the reactive metabolite).

A human liver microsomal (HL-mix-13) incubation mixture containing either 100 or 10 μM substrate (acetaminophen), P450 concentration equivalent to 0.5 μM P450, 1 mM glutathione (GSH), and 100 mM potassium phosphate buffer (pH 7.4) was pre-incubated for 3 minutes at 37° C. The reaction was initiated by the addition of an NADPH-generating system (0.54 mM NADP+, 11.5 mM MgCl₂, 6.2 mM DL-isocitric acid, and 0.5 Units/ml (U/ml) isocitric dehydrogenase). The final incubation volume was 250 μL. Samples without functional NADPH-generating system were used as negative controls. After 30 minutes incubation at 37° C., 375 μL of acetonitrile was added to the incubation mixture, which was then centrifuged at 3,500 rpm (revolutions per minute) for 10 minutes. The supernatant was transferred to a 96-well microwell plate and placed in a nitrogen evaporator (Evaporex EVX-192, Apricot Designs, Monrovia, Calif., US) to reduce the percent organic prior to solid phase extraction.

For assays run using glutathione ethyl ester, a human liver microsomal (HL-mix-13) incubation mixture containing either 250 or 10 μM substrate, P450 concentration equivalent to 0.5 μM P450, 1 mM glutathione ethyl ester (GSH-EE), and 100 mM potassium phosphate buffer (pH 7.4) was pre-incubated for 3 minutes at 37° C. The reaction was initiated by the addition of an NADPH-generating system (0.54 mM NADP⁺, 11.5 mM MgCl₂, 6.2 mM DL-isocitric acid, and 0.5 Units/ml (U/ml) isocitric dehydrogenase). The final incubation volume was 250 μL. Samples without a functional NADPH— generating system were used as negative controls. After 30 minutes incubation at 37° C., 375 μL of acetonitrile was added to the incubation mixture, which was then centrifuged at 3,500 rpm (revolutions per minute) for 10 minutes. The supernatant was transferred to a 96-well microwell plate and placed in a nitrogen evaporator (Evaporex EVX-192, Apricot Designs, Monrovia, Calif., US) to reduce the percent organic prior to solid phase extraction.

Glutathione-drug conjugates and/or glutathione ethyl ester-drug conjugates were extracted from the microwells using a Waters Oasis HLB 96-well microelution plate (although a standard plate could be used, a microelution plate is preferred because of the reduced time in performing the solid phase extraction using the microelution plate). A 384-channel Personal-150 pipettor fitted with a 96 channel head (Apricot Designs Inc., Monrovia, Calif., US) was used during SPE (solid phase extraction) to facilitate solvent transfer. The SPE plates were conditioned by passing 200 μL methanol followed by 200 μL water through each of the SPE cartridges. To initiate solvent flow through the sorbent, vacuum was applied to the receiving side of the SPE plate using a 96-well extraction manifold (Tomtec Inc., Hampden, Conn., US). Microsomal incubation samples were added to the 96-well plate and washed with 200 μL water. Analyte was desorbed using 50 μL 40:60 (v/v %) acetonitrile: isopropyl alcohol. The desorption solvent was collected in a 96-well plate fitted with 0.7 mL (milliliters) low retention/conical glass tubes that are designed for injecting low sample volumes (Waters Corp., Milford, Mass., US). Solvent was evaporated using a 96-channel Evaporex EVX-192 evaporator (Apricot Designs, Monrovia, Calif., US) using nitrogen as a drying gas. Each sample was reconstituted with 50 μL starting mobile phase composition (5:95 (v/v %) acetonitrile:water 0.5% formic acid/5 mM ammonium formate).

An Agilent 1100 capillary liquid chromatography system (Agilent, Palo Alto, Calif., US) was used. The autosampler module was configured to inject sample from 96-well plates. Chromatography was performed in a Vydac 300 μm (micrometers) I.D. (inner diameter)×5 cm (centimeters) (long) C₁₈ column (a microbore column) that contained 5 μm particles with a pore size of 300 Angstroms (Grace Vydac, Hesperia, Calif., US). The tubing connecting the injection port to the analytical column and analytical column to ESI (electrospray ionization) source housing was made of PEEK (poly-ether-ether-ketone resin) and had a I.D. of 75 μm and O.D. (outer diameter) of 365 μm.

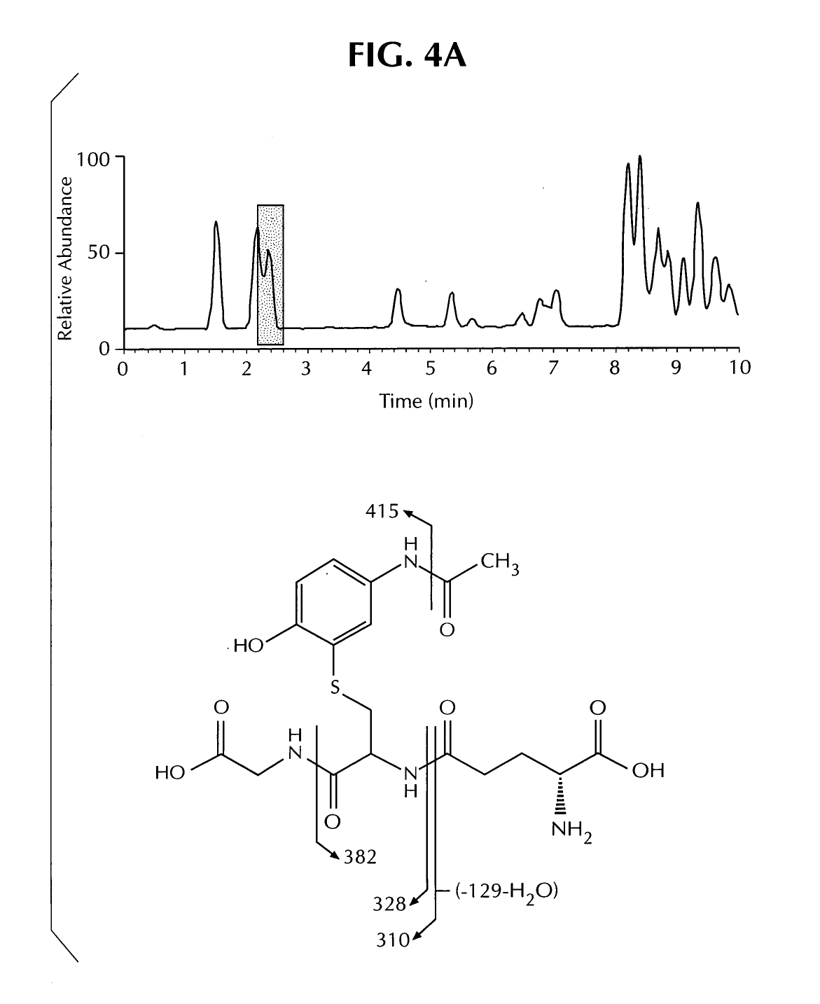

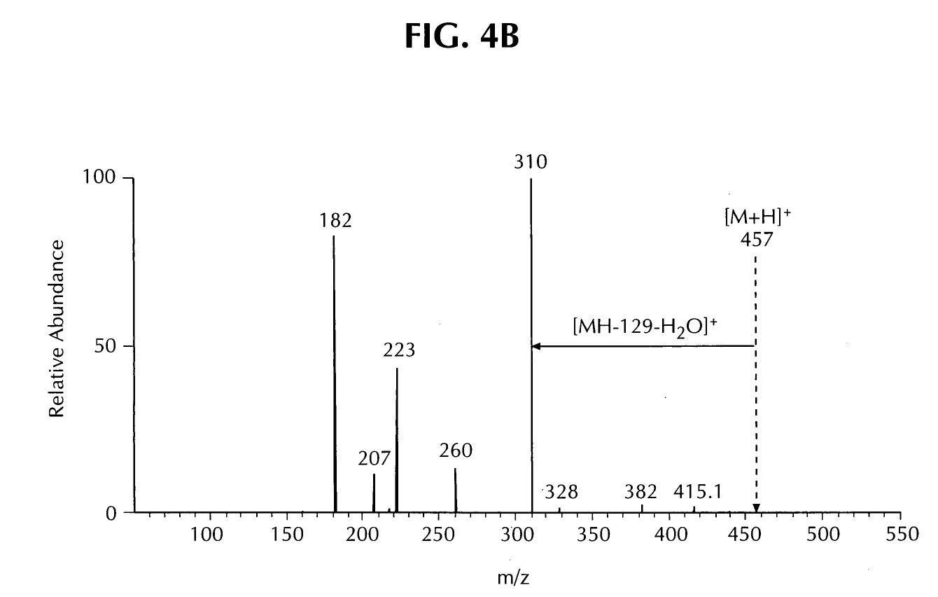

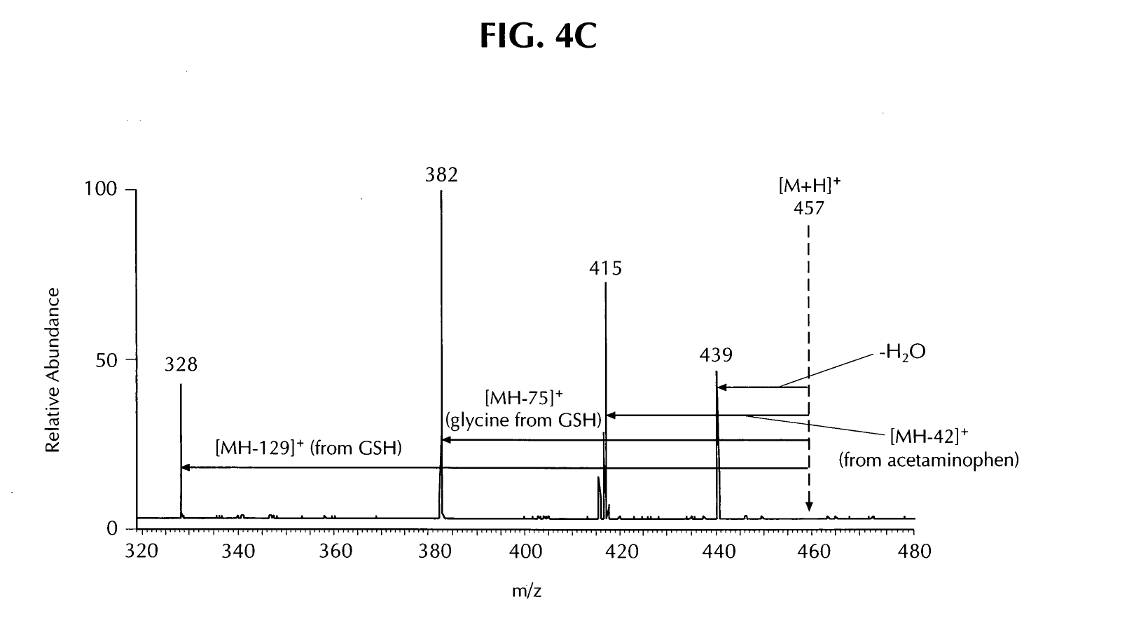

Glutathione-acetaminophen (FIG. 4) was isolated from endogenous sample components using a binary mobile phase (MP) system comprising 0.2% acetic acid and 0.05% trifluoroacetic acid in water (MP A) and 0.2% acetic acid and 0.05% trifluoroacetic acid in acetonitrile (MP B) at a flow rate of 5 μL/minute. For all other experiments, the respective compound and/or conjugates were isolated from endogenous sample components using a binary mobile phase (MP) system comprising 5 mM (millimolar) ammonium formate and 0.05% formic acid in water (MP A) and 5 mM (millimolar) ammonium formate and 0.05% formic acid in acetonitrile (MP B). For both the trifluoroacetic acid MP system and for the ammonium formate MP system, following injection of a 0.5 μL sample, a MP composition of 95% A-5% B was held isocratic for 5 minutes. From 5 to 10 minutes, the % MP B was increased linearly from 5 to 95. At 10 minutes, the % MP B was returned to 5% and the column allowed to re-equilibrate for 5 minutes prior to the next injection (total cycle time/analysis=15 minutes).

A Thermo-Finnigan orthogonal electrospray ionization interface and Quantum triple quadrupole mass spectrometer (Thermo-Finnigan Corp., San Jose, Calif., US) were used. A bare fused silica (BFS) capillary (100 μm I.D.×190 μm O.D.) was used to transfer chromatographic effluent from the zero dead volume union on the ESI source housing to the orthogonal ESI probe assembly. A 34 gauge stainless steel needle (i.e., a microbore needle) was used in place of the standard bare fused silica ESI capillary to achieve efficient and stable microelectrospray ionization (referred to herein as μESI or ESI). ESI was initiated by applying voltage of 3.5 kV (positive polarity) to the stainless steel needle. ESI spray stability was enhanced using a sheath gas (nitrogen) setting of 10 psi (pounds per square inch). The auxiliary gas pressure and source transfer capillary temperature were maintained at 5 psi and 250° C., respectively. The ESI probe was held at a position that placed it as close to 90° from the entrance of the sweep cone as possible.

Optimal tandem MS parameters for glutathione conjugates were established using a 1 μM s-(p-nitrobenzyl)-glutathione (s-p-NBGSH) standard solution. Operating the mass spectrometer in product ion mode (Q1 transmits [M+H]⁺m/z 443 while Q3 scans from 100 to 500 Da), the efficient loss of the characteristic 129 Da neutral fragment from the glutathione moiety of s-p-NBGSH was achieved using a Q2 offset voltage of −16V while maintaining the Q2 cell pressure at 1.0 mtorr (millitorr).

Optimal tandem MS parameters for glutathione ethyl ester conjugates were established using a 1 μM s-(p-nitrobenzyl)-glutathione ethyl ester (s-p-NBGSH-EE) standard solution. Operating the mass spectrometer in product ion mode (Q1 transmits [M+H]⁺m/z 471 while Q3 scans from 100 to 500 Da), the efficient loss of the characteristic 129 Da neutral fragment from the glutathione ethyl ester moiety of s-p-NBGSH-EE was achieved using a Q2 offset voltage of −16V while maintaining the Q2 cell pressure at 1.0 mtorr (millitorr).

Two tandem scanning techniques, constant neutral loss (CNL) and selected reaction monitoring (SRM), were employed during in vitro sample analysis to detect glutathione-acetaminophen conjugates (FIG. 4). While operating in CNL scan mode, Q1 was set to scan over the mass range 350 to 550 Da (1-second scan time) while Q3 (linked to Q1) scanned the mass range 221 to 521 Da. When SRM was used, Q1 was set to transmit the [M+H]⁺ ion of the acetaminophen-glutathione conjugate (m/z 457) while Q3 transmitted the product ion (m/z 328, 0.5-second scan time, +/−0.2 Da scan window) resulting from the neutral loss of 129 Da from the parent [M+H]⁺ ion.

A single scanning technique, selected reaction monitoring (SRM), was employed during in vitro analysis to detect glutathione ethyl ester conjugates, s-(p-nitrobenzyl)-glutathione or s-(p-nitrobenzyl)-glutathione ethyl ester, and all experiments using the assay substrate test set (for both glutathione and for glutathione ethyl ester). In SRM, Q1 was set to transmit the [M+H]+ion of the respective conjugate or standard as the case may be, while Q3 transmitted the product ion 0.5-second scan time, +/−0.2 Da scan window) resulting from the neutral loss of 129 Da from the respective parent [M+H] ion.

A Microsoft Excel® macro was used to generate a table of prospective selected reaction monitoring (SRM) transitions for each substrate in the assay substrate test set. This was referred to as the multiple reaction monitoring battery calculation (MRM). The MRM table was imported into template MS methods and the final SRM scanning method that was used during the MS/MS analysis. The calculated MH⁺ masses for each compound were based on potential metabolic changes to the parent substrate structure as a result of characteristic bioactivation pathways that lead to reactive intermediate formation and subsequent conjugation with either glutathione or glutathione ethyl ester. Since the intended use of the macro would be to generate MRM tables for a large number of substrates, the macro calculations covered a broad class of molecular structures so as to minimize the possibility of not detecting reactive intermediate formation due to incorrect mass-to-charge (m/z) monitoring when performing the selected reaction monitoring (SRM) analysis.

FIG. 3

A product ion spectrum of the [M+H]⁺ ion of s-p-NBGSH is shown in FIG. 3. Three of the prominent product peaks represent ions that are formed following the loss of neutral fragments from the glutathione moiety. The ions at m/z (mass/charge) 368 ([MH-75]⁺) and m/z 314 ([MH-129]⁺) represent the loss of glycine and pyroglutamate, respectively, while the prominent ion at m/z 296 represents the loss of pyroglutamate and a water molecule. Tandem mass spectrometry collision induced dissociation (CID) parameters were optimized to maximize the efficiency of parent ([M+H]⁺=443) to product ion ([MH-129]⁺=314) transition. The largest abundance of [MH-129]⁺ productions were formed using a collision offset voltage of −16V and a collision cell pressure of 1.0 mtorr (FIG. 3). Increasing the offset voltage and cell pressure above those values resulted in an increase in the abundance of [MH-75]⁺ ions and the daughter ion at m/z 211; however, the abundance of those ions did not surpass the levels observed for the [MH-129]⁺ ion using the optimized tandem mass spectrometry parameters.

FIG. 4

FIG. 4A shows the HPLC/product ion scan (high performance liquid chromatography/product ion scan) analysis of microsomal incubation samples (1 mM GSH, NADPH generating system. 0.5 uM P450 and 100 uM potassium phosphate) dosed with acetaminophen at 100 uM. The total ion chromatogram shows a prominent peak at RT of approximately 2.3 minutes. The most prominent peak in product ion spectrum FIG. 4B (m/z 310) is the ion resulting from the neutral loss of pyroglutamate and a water molecule (−147 Da) from the parent acetaminophen-GSH conjugate (m/z 457). FIG. 4C shows the less abundant product ions that are characteristic of an acetaminophen-GSH conjugate. The ions at m/z 328 and 382 represent the loss of pyroglutamate and glycine, respectively, from the GSH moiety. The ions at m/z 415 and 439 represent neutral losses that are characteristic for the acetaminophen moiety.

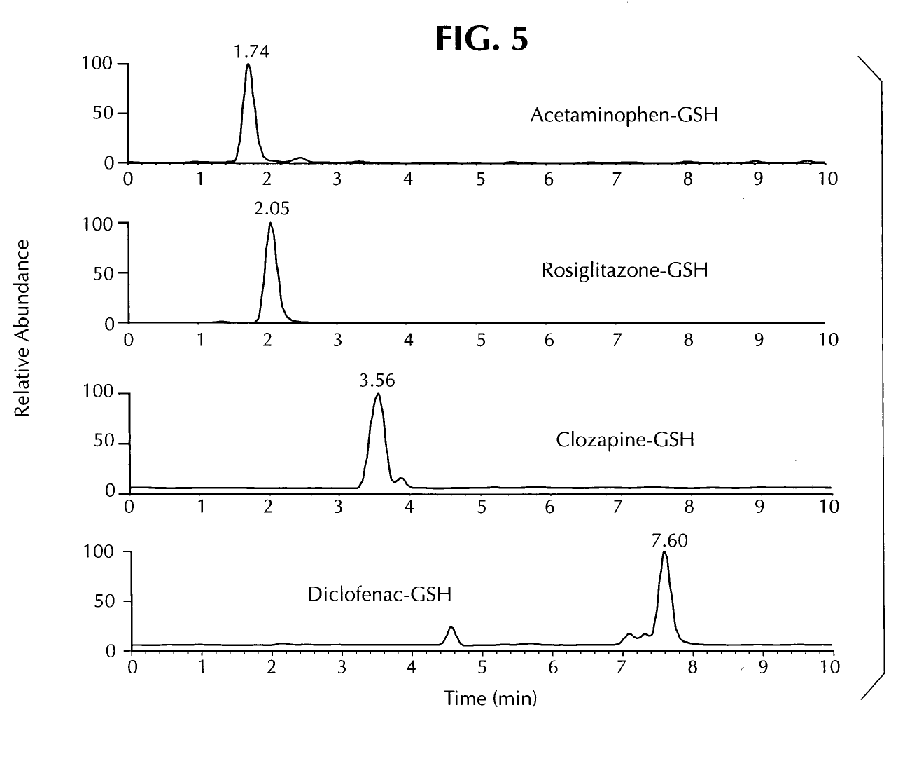

FIG. 5

An assay substrate test set was used to assess the detection capabilities of the methods of the invention. This twelve substrate test set is shown in Table 1. This assay substrate test set was chosen based on literature reports, structural diversity and diversity in bioactivation mechanisms. Of the twelve substrates in the test set, ten substrates have been reported in the literature to cause idiosyncratic toxicity and form reactive intermediates, while the other two do not cause idiosyncratic reactions, but do form reactive intermediates. Since all of the validation substrates form reactive intermediates and could be trapped using a conjugating species, the detection capability of the methods of the invention using glutathione and glutathione ethyl ester was assessed based on the number of positive responses observed upon incubation with each of the twelve substrates of the substrate set. A response in the assay was deemed positive if there were no co-eluting species observed in control (no NADPH-generating components) samples and analyte peak signal to noise ratio was >10.

TABLE 1

| | Bioactivation | | |
|---|---|---|---|
| | Structure | | Reactive |
| Substrate | Class | Structure | Imtermediate |
| Acetaminophen^{P} | Masked aniline | 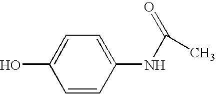 | Guinone-imine |
| Clozapine^{P} | Fused azaheterocycle | 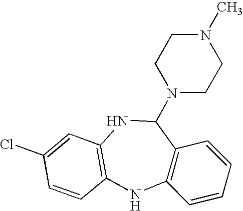 | Nitrenium ion |
| Amodiaquine^{P} | Masked aniline | 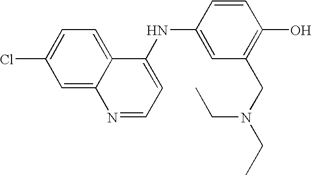 | Quinone-imine |
| Diclofenac^{P} | Aniline derivative | 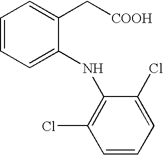 | Quinone-imine |
| Rosiglitazone^{N} | Glitazone | 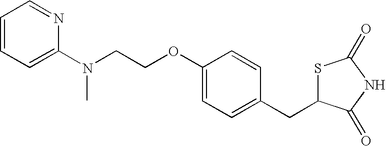 | α-keto- isocyanate |
| Indomethacin^{P} | Indole | 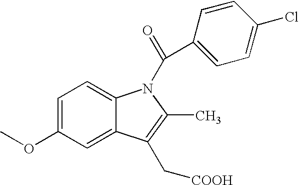 | Iminoquinone |
| Sulfamethoxazole^{P} | Aniline | 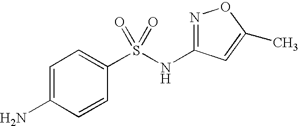 | Nitrosoamine |
| Carbamazepine^{P} | Benzene rings | 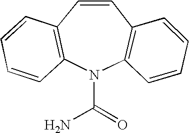 | Arene oxide |
| Felbamate^{P} | Michael Acceptor | 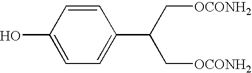 | Atropaldehyde |
| Pioglitazone^{N} | Glitazone | 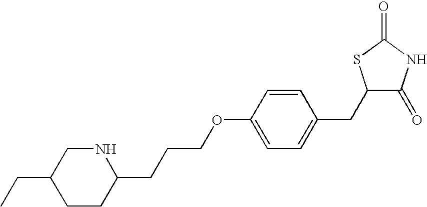 | α-keto- isocyanate |
| Imipramine^{P} | Benzene rings | 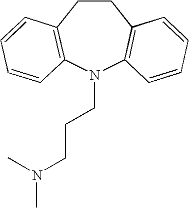 | Arene oxide |
| Valproic acid^{P} | Fatty acids | 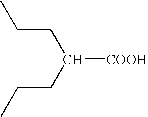 | α-β- unsaturated- carbonyl |

The detection capabilities of the assay were assessed using GSH as the compound in the methods of the invention using a substrate concentration of 10 μM for the substrates in the assay substrate test set. Of the twelve substrates in the assay substrate test set, four were detected as being conjugated by GSH following microsomal incubation; in other words, the four had positive responses in the assay. HPLC/SRM (high performance liquid chromatograph/selected reaction monitoring) analysis was performed of microsomal incubation samples (1 mM GSH, NADPH generating system, 0.5 μM P450 and 100 μM potassium phosphate) dosed with 10 μM substrate. FIG. 5 shows the traces of the positive responses using GSH and the assay substrate test set. The total ion chromatograms show a prominent peak for each of the four positive responses, with the acetaminophen-GSH peak migrating at an RT of approximately 1.74 minutes, the rosiglitazone-GSH peak migrating at an RT of approximately 2.05 minutes, the clozapine-GSH peak migrating at an RT of approximately 3.56 minutes and the diclofenac-GSH peak migrating at an RT of approximately 7.60 minutes.

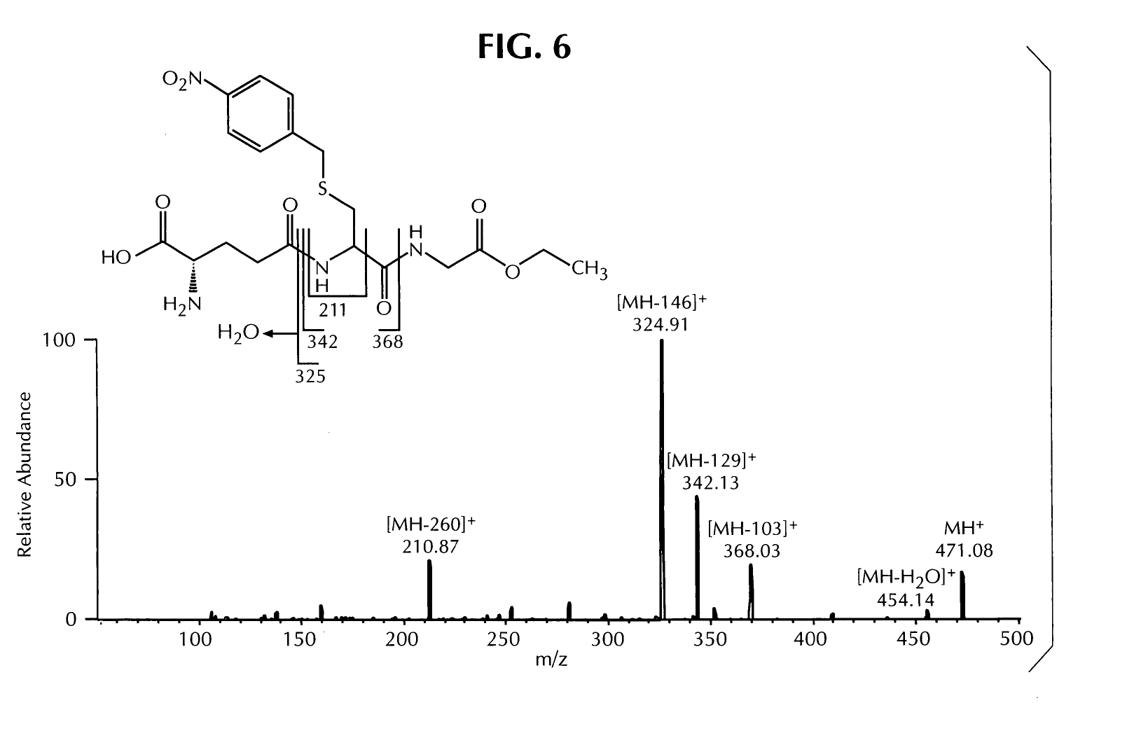

It was decided to test another compound in the assays of the invention to determine whether a different reactive compound would result in a greater number of positive responses with the assay substrate test set. The close-in analog of glutathione, glutathione ethyl ester was identified as an alternative compound. FIGS. 6-8 describe the characterization of that compound in the assays of the invention.

FIG. 6

A product ion spectrum of the [M+H]⁺ ion of s-(p-nitrobenzyl)-glutathione ethyl ester is shown in FIG. 6. Three of the prominent product peaks represent ions that are formed following the loss of neutral fragments from the glutathione ethyl ester moiety. The spectrum contains ions representing neutral loss of 129 and 146 u (u equals a unit, or 1 atomic mass unit (1 amu)) from the parent ion at m/z 471, shows an ion at m/z 368 ([MH-103]⁺) that results from the loss of the glycylethylester portion of the moiety. Tandem mass spectrometry collision induced dissociation (CID) parameters were optimized to maximize the efficiency of parent ([M+H]+=471) to product ion ([MH-129]⁺) transition. The largest abundance of [MH-129]⁺ product ions were formed using a collision offset voltage of −16V and a collision cell pressure of 1.0 mtorr (FIG. 6). Increasing the offset voltage and cell pressure above those values resulted in an increase in the abundance of [MH-103]+ ions and the daughter ion at 325 m/z.

FIG. 7

An s-p-NBGSH standard curve (diluent was 5:95 (v/v %) acetonitrile:water 0.05% formic acid/5 mM ammonium formate) and an s-p-NBGSH-EE standard curve (diluent was 5:95 (v/v %) acetonitrile:water 0.05% formic acid/5 mM ammonium formate) were prepared. The concentration range of both standard curves was identical and covered approximately two orders of magnitude from 5 nM to 300 nM. The diluent used to prepare the standard curves was the same as the solution used to reconstitute samples following sample preparation (SPE). Both standard curves were run sequentially and in duplicate. The resulting peak areas for both s-p-NBGSH and s-p-NBGSH-EE were plotted versus concentration and the results shown in FIG. 7. Least squares regression was used to generate the best-fit line and mathematical equation (y=m×+b) adjacent to each curve. Evident in the equations is an approximate 10-fold increase in slope equates to a 10-fold increase in MS sensitivity for s-p-NBGSH-EE versus s-p-NBGSH. The 10-fold increase in MS sensitivity was confirmed by infusing equi-molar/same solvent solutions of s-p-NB-GSH-EE and s-p-NB-GSH and comparing MH+ ion signal intensities (data not shown).

An s-p-NBGSH standard curve (diluent was 5:95 (v/v %) acetonitrile:water 0.05% formic acid/5 mM ammonium formate) was prepared and analyzed to establish LOQ (limit of quantitation) and LOD (limit of detection). Based on s-p-NBGSH peak area suing selected reaction monitoring (SRM), the LOQ of the assay (10 times signal to noise) was 5.0 fmoles (femtomoles) (on column) while the LOD (3 times signal to noise) was 1.5 fmoles (on column). The analytical assay showed good linearity over a concentration range of 5 nM to 300 nM. To estimate the losses that would occur following SPE, a 200 ng/mL s-p-NBGSH serial dilution sample from the standard curve was extracted following the SPE procedure described above. Based on peak area comparison (extracted ion), the analyte recovery was approximately 85%.

An s-p-NBGSH-EE standard curve (diluent was 5:95 (v/v %) acetonitrile:water 0.05% formic acid/5 mM ammonium formate) was prepared and analyzed to establish LOQ (limit of quantitation) and LOD (limit of detection). Based on s-p-NBGSH-EE peak area selected reaction monitoring (SRM), the LOQ of the assay (10 times signal to noise) was 0.57 fmoles (femtomoles) (on column) while the LOD (3 times signal to noise) was 0.17 fmoles (on column). The analytical assay showed good linearity over a concentration range of 5 nM to 300 nM.

It was concluded from this that the assay is 10-fold more sensitive using GSH-EE as the compound that reacts with the reactive species which are generated.

FIG. 8

FIG. 8 shows the HPLC (high performance liquid chromatography) analysis of the standards s-p-NB-GSH and s-p-NB-GSH-EE. The total ion chromatogram in trace A shows a prominent peak for s-p-NB-GSH at a retention time (RT) of approximately 3.9 minutes. The total ion chromatogram in trace B shows a prominent peak for s-p-NB-GSH-EE at a RTof approximately 8.2 minutes. This apparent difference in hydrophobicity was anticipated, as discussed above, due to the reduction in acidic sites from two carboxylic acids on glutathione, to one carboxylic acid site on glutathione ethyl ester. The observed increase hydrophobicity of s-p-NBGSH-EE when compared to that of s-p-NBGSH, was verified by the observation of an increase chromatographic retention time for the respective compound—acetaminophen conjugates, acetaminophen-GSH-EE and acetaminophen-GSH (data not shown).

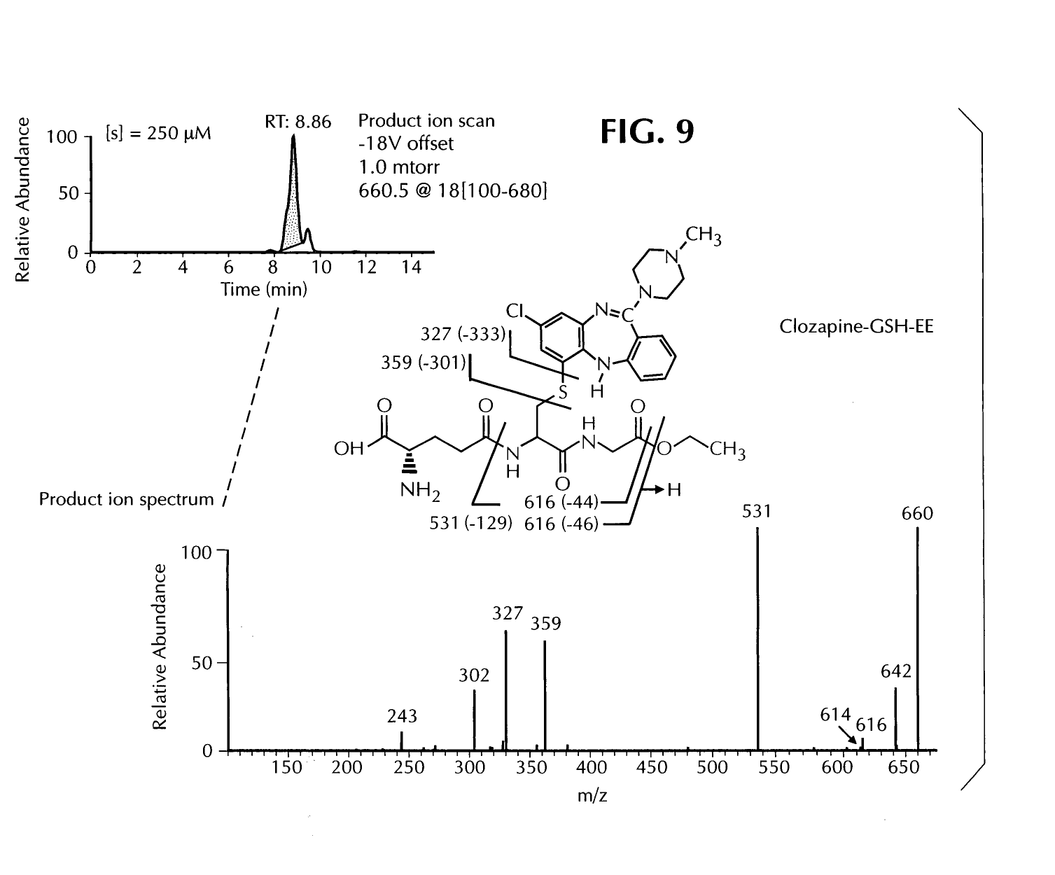

FIG. 9

FIG. 9 shows the HPLC/product ion scan (high performance liquid chromatography/product ion scan) analysis of microsomal incubation samples (1 mM GSH-EE, NADPH generating system, 0.5 μM P450 and 100 μM potassium phosphate) dosed with clozapine at 250 μM. The total ion chromatogram shows a prominent peak at RT of approximately 8.9 minutes. The most prominent peaks in the product ion scan are the parent ion [MH⁺] (m/z 660.5) and the product ion resulting from the characteristic loss of pyroglutamate [MH-129] (m/z 531).

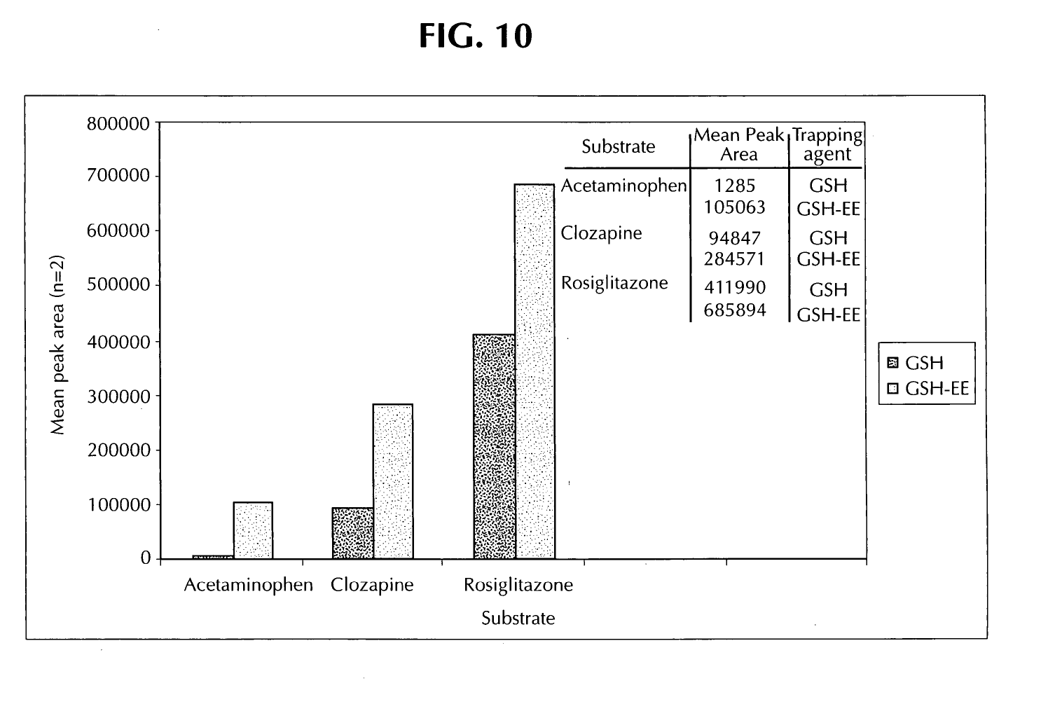

FIG. 10

A separate study using the substrates that gave a positive response in the assay using GSH (acetaminophen, clozapine, rosiglitazone) out of the twelve substrates in the assay substrate test set was conducted with microsomal incubation samples (1 mM either GSH or GSH-EE, NADPH generating system, 0.5 μM P450 and 100μ potassium phosphate) dosed with 10 μM substrate. FIG. 10 shows the results of the study in terms of increase in selected reaction monitoring (SRM) analysis response using GSH-EE as the compound versus using GSH as the compound. The graph in FIG. 10 represents the mean peak area obtained in a total ion chromatogram with each different substrate using either glutathione or glutathione ethyl ester as the compound. As is evident from FIG. 10, a minimum of two-fold increase in peak area was observed using GSH-EE as the trapping agent. For compounds such as acetaminophen, the increase in conjugate peak area was greater than ten fold.

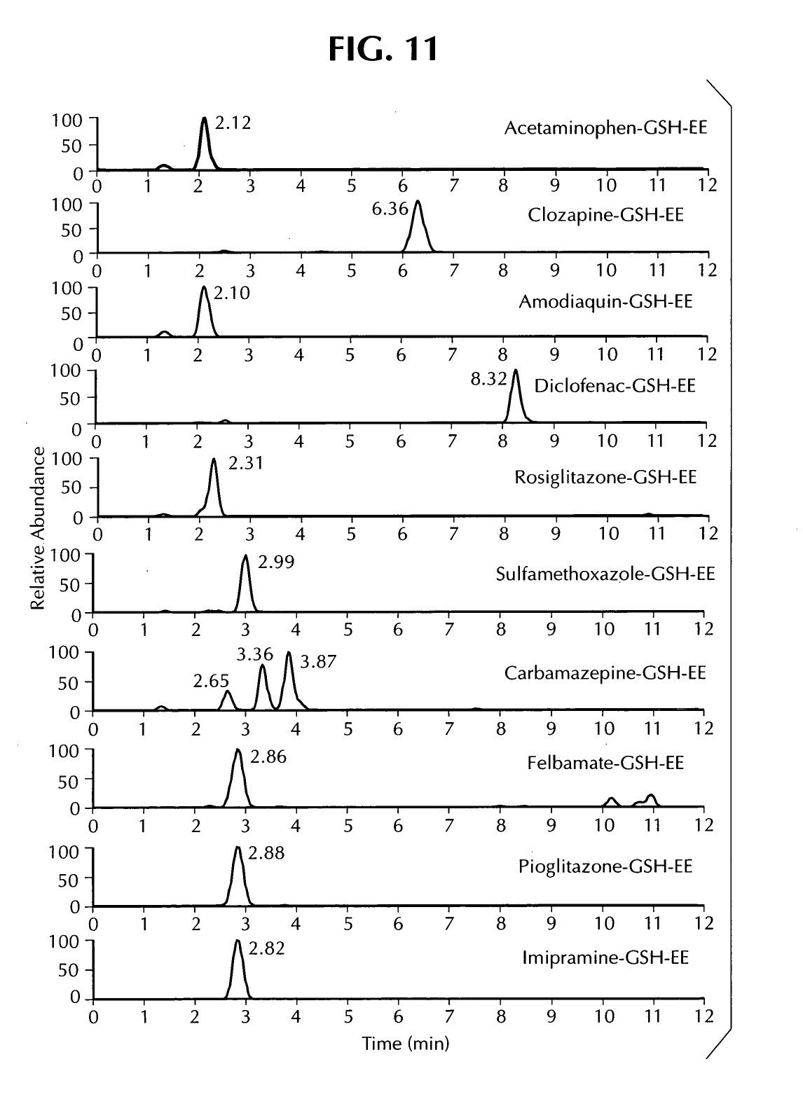

FIG. 11

The detection capabilities of the assays of the invention were then assessed using GSH-EE as the compound using a substrate concentration of 10 μM. Of the twelve substrates in the assay substrate test set, ten gave positive responses in the assay, or were detected as being conjugated by GSH-EE following microsomal incubation (Table 2). HPLC/SRM (high performance liquid chromatography/selected reaction monitoring) analysis was performed of microsomal incubation samples (1 mM GSH-EE, NADPH generating system, 0.5 μM P450 and 100 μM potassium phosphate) dosed with 10 uM substrate. FIG. 11 shows the traces of the total ion chromatograms of ten substrates that gave a positive response using GSH-EE and the assay substrate test set. The total ion chromatograms show a prominent peak for each of the ten positive responses, with the acetaminophen-GSH-EE peak migrating at an RT of approximately 2.12 minutes, the clozapine-GSH-EE peak migrating at an RT of approximately 6.36 minutes, the amodiaquin-GSH-EE peak migrating at an RT of approximately 2.10 minutes, the diclofenac-GSH-EE peak migrating at an RT of approximately 8.32 minutes, the rosiglitazone-GSH-EE peak migrating at an RT of approximately 2.31 minutes, the sulfamethosazole-GSH-EE peak migrating at an RT of approximately 2.99 minutes, the carbamazepine-GSH-EE conjugates having three peaks migrating at RTs of approximately 2.65, 3.36 and 3.87 minutes, the felbamate-GSH-EE peak migrating at an RT of approximately 2.86 minutes, the pioglitazone-GSH-EE peak migrating at an RT of approximately 2.88 minutes, and the imipramine-GSH-EE peak migrating at an RT of approximately 2.82 minutes.

TABLE 2

| | Response observed using | Response observed using |
|---|---|---|
| Substrate | GSH as trapping agent | GSH-EE as trapping agent |
| Acetaminophen | Yes | Yes |
| Clozapine | Yes | Yes |
| Amodiaquine | No | Yes |
| Diclofenac | Yes | Yes |
| Rosiglitazone | Yes | Yes |
| Indomethacin | No | No |
| Sulfamethoxazole | No | Yes |
| Carbamazepine | No | Yes |
| Felbamate | No | Yes |
| Pioglitazone | No | Yes |
| Imipramine | No | Yes |
| Valproic acid | No | No |

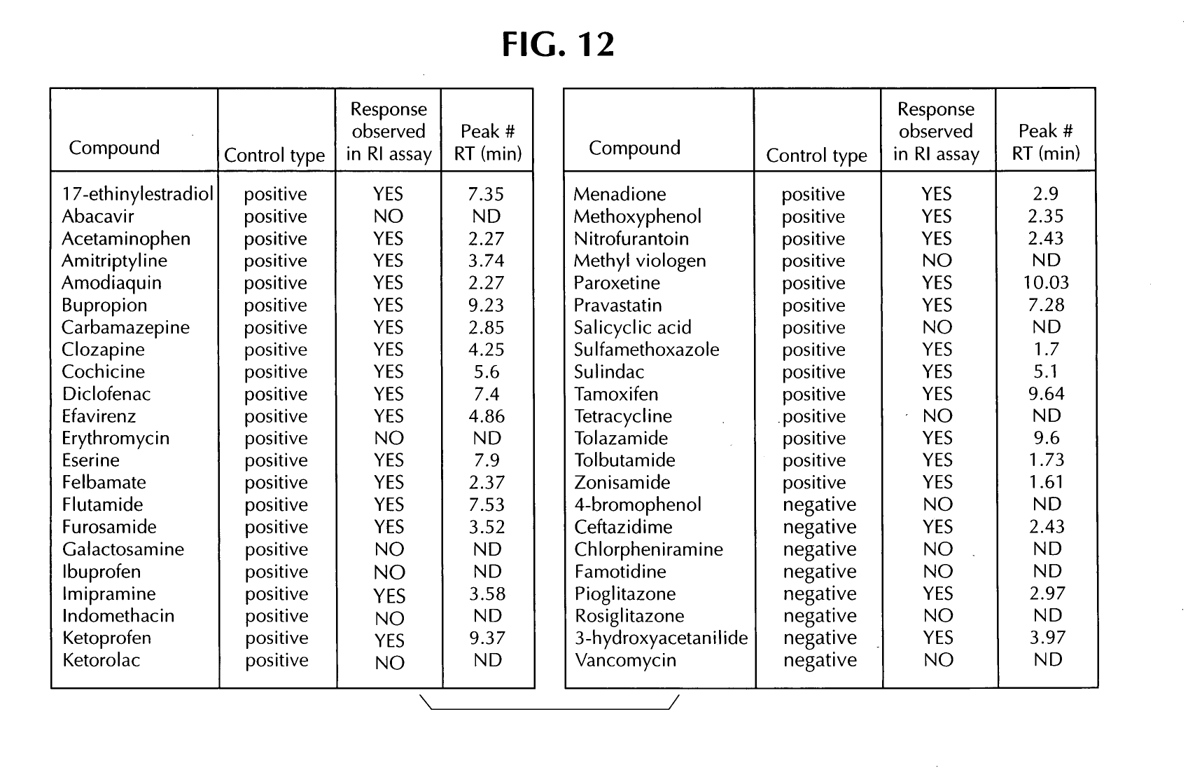

FIG. 12

The activity of glutathione ethyl ester in the assay was tested using the twelve substrates set forth in Table 1, as well as 38 other substrates. These 50 substrates were known to give either positive or negative responses in a test for human hepatotoxicity as described in the literature. Those substrates that were known to show human hepatotoxicity in a literature assay are designated “positive” in FIG. 12, while those that did not show human hepatotoxicity in literature reports are designated “negative” in FIG. 12. HPLC/SRM (high performance liquid chromatography/selected reaction monitoring) analysis was performed of microsomal incubation samples (1 mM GSH-EE, NADPH generating system, 0.5 μM P450 and 100 μM potassium phosphate) dosed with 10 μM substrate. N=3. FIG. 12 shows the results of the analysis with “Yes” indicating a positive response in the assays of the invention, and “No” indicating a negative response in the assays of the invention. A response in the assay was deemed “yes” if there were no co-eluting species contained in control (blank) samples and analyte peak to signal ratio was >10. A response in the assay was deemed “no” if the signal to noise ratio was <10. A “no” in the assay does not necessarily mean that conjugates are not present, but that, if present, the conjugate(s)' abundance would not result in a signal to noise ratio>10.

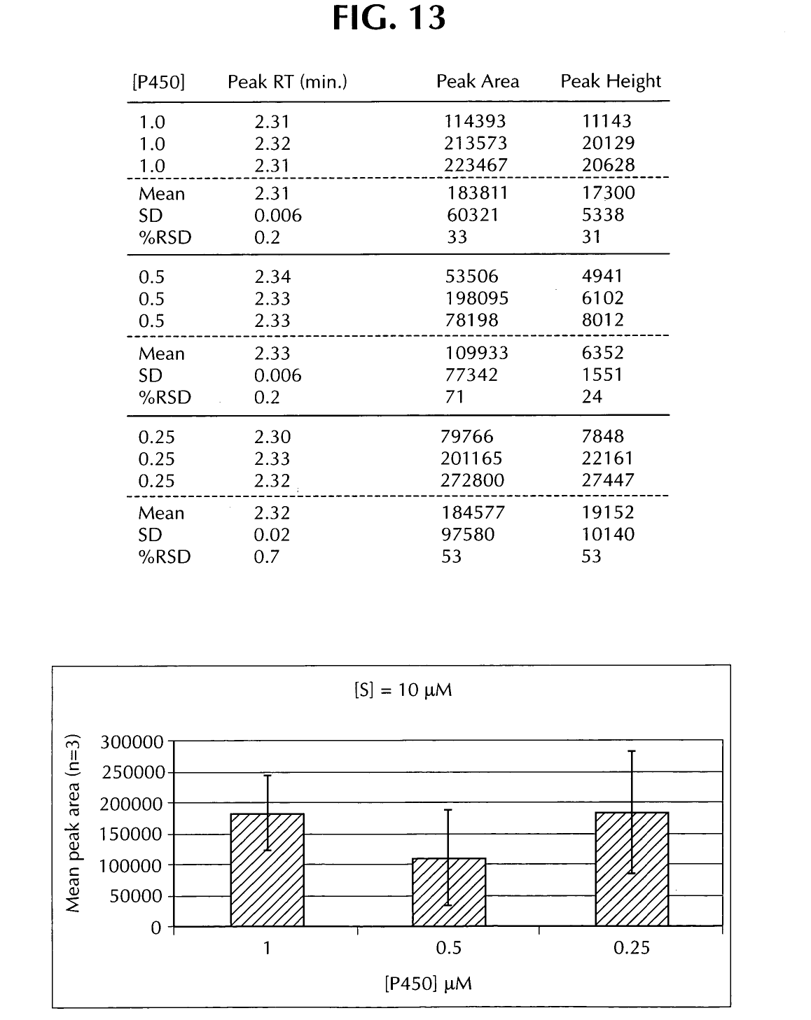

FIG. 13

SRM was used to determine if P450 concentration had an effect on acetaminophen reactive metabolite (quinone-imine intermediate) formation in vitro (see FIG. 2 regarding metabolic pathways). Three different concentrations of P450 enzyme (1, 0.5, and 0.25 μM) were used in the microsomal assay and the resultant acetaminophen-GSH conjugate levels determined. FIG. 13 shows a summary of the acetaminophen-GSH peak data using each P450 concentration. As is evident, P450 concentration did not have a significant affect on acetaminophen quinone-imine formation. These data show that a P450 concentration of 0.25 μM can be used in the in vitro assay to induce reactive metabolite formation and that the analytical method is capable of detecting the resulting reactive metabolite levels.

The low detection limits are made possible in part by the use of microbore column liquid chromatography, microelectrospray ionization, and tandem mass spectrometry scanning techniques described above. In FIG. 4B, the prominent transition appeared to be the loss of the 147 Da neutral fragment ([MH-129-18]⁺) from the parent conjugate. If this transition appears as the prominent transition when analyzing GSH conjugates in the future, then monitoring for this neutral loss may provide a means separate and apart from the other means discussed to further reduce the LOD and LOQ.

The preferred low solvent flow rate used takes advantage of the increase in analyte ionization efficiency when performing ESI at μL and nL/minute flow rates. The benefit of this is the reason its use is so prevalent in the mass spectrometry literature. The use of a 34 gauge stainless steel needle instead of the standard BFS (bare fused silica) capillary tubing during PESI also contributes to decreasing the LOD and LOQ. In fact, the use of this PESI setup has been shown to achieve LOD comparable to those attained while operating at flow rates commonly used during capillary liquid chromatography (e.g., 200 nL/min.). PESI spray stability was also improved by using the stainless steel needle instead of the BFS capillary.

Optimizing the collision cell parameters to maximize the loss of the 129 Da neutral fragment from the glutathione ethyl ester moiety is also beneficial in reducing LOD and LOQ. The use of these parameters in conjunction with constant neutral loss scanning enables the detection of 0.34 nM (nanomolar) s-p-NBGSH-EE (selected reaction monitoring). The benefit of using CNL scanning to screen for reactive metabolite formation is that the molecular weight of the conjugates (the moieties formed by the reaction of the reactive species and the compound that reacts with them) does not have to be known prior to analysis. Thus, for example, CNL mode is particularly useful if a compound undergoes extensive metabolism to form a reactive metabolite that has a molecular weight differing substantially from the parent compound prior to conjugation with glutathione or another compound. Additional studies can then be performed to identify metabolite structure, sites of metabolism, etc. It is expected that CNL scanning will typically cover a molecular weight range of about 53 to about 553, which requires a scanning range of about 360 to about 860 m/z if glutathione is used (the molecular weight of glutathione is about 307); however, other compounds that bind with the reactive species and other substrate molecular weights may be used. Thus, the expected preferred range of 360 to 860 could be modified accordingly.

SRM mode is considered to be a more sensitive scanning technique than CNL because the signal-to noise-level for the analyte(s) of interest is increased due to a reduction in background noise. When using SRM mode, the masses of both parent and product ion must be known prior to analysis. Therefore, this technique is useful for targeted analysis (detection) of reactive metabolites.

The experiments that were conducted using acetaminophen, clozapine and the assay substrate test set illustrate the specificity and sensitivity of the methods of this invention. It is expected that the method of this invention will also enable screening of a broad range of compounds (substrates) for several reasons, including the exceedingly low LOD that is possible, thereby allowing very low concentrations of substrates to be used and thereby essentially removing solubility as a potential problem even with the wide range of compounds (substrates) that are expected to be subjected to the method of this invention.

A further advantage of using the in vitro assays of this invention to screen a wide variety of compounds for reactive metabolite formation (and, therefore, for possible toxicity) was shown by the ability to form and then detect acetaminophen-glutathione conjugates using acetaminophen (substrate) concentrations that would minimize solubility issues by coming closer to in vivo substrate levels than other assays. This further advantage was also shown by the ability to form and to detect clozapine-glutathione ethyl ester conjugates using clozapine (substrate) concentrations that would minimize solubility issues by coming closer to in vitro substrate levels than other assays. In other words, the in vitro methods of this invention come closer to mimicking in vivo conditions in the biology phase (i.e., in the production of reactive species), and, as those of ordinary skill in the art will appreciate, this makes the results of the assay relevant and, therefore, makes the assay valuable (e.g., as a predictive tool for possible in vivo toxicity).

Variations and modifications will be apparent to those skilled in the art and the claims are intended to cover all variations and modifications falling within the true spirit and scope of the invention. Thus, for example, the method of this invention need not result in just yes-no (positive-negative) answers regarding the possibility of in vivo toxicity for particular compounds. The data/results may also be used to establish a structure-activity relationship and be used as a predictive tool by chemists regarding the effects of changing a molecule's structural features (e.g., pendant groups).

1. An in vitro method for detecting one or more reactive species that may be formed from a substrate by an enzyme system, the method comprising:
(a) contacting in vitro in a first mixture the substrate with a substrate metabolizing enzyme system whereby one or more reactive species may be formed, the concentration of the substrate in the first mixture being no greater than about 50 μM;
(b) contacting in vitro the one or more reactive species, if formed, with a compound that reacts with the one or more reactive species to form one or more detectable species; and
(c) detecting at least some of the one or more detectable species formed in step (b).

2. The method of claim 1 wherein the contacting of step (b) occurs in the first mixture.

3. The method of claim 2 further comprising assessing the presence of the one or more reactive species based on the results of step (c).

4. The method of claim 1 wherein the substrate metabolizing enzyme system is normally present in vivo.

5. The method of claim 4 wherein the substrate metabolizing enzyme system comprises a microsomal substrate metabolizing enzyme system.

6. The method of claim 5 wherein the substrate metabolizing enzyme system comprises a P450 system.

7. The method of claim 4 wherein the compound comprises a compound normally present in vivo.

8. The method of claim 7 wherein the compound comprises a compound that normally reacts with reactive species in vivo.

9. The method of claim 6 wherein the compound comprises glutathione ethyl ester.

10. The method of claim 9 wherein the one or more detectable species comprise a glutathione ethyl ester conjugate.

11. The method of claim 10 wherein step (c) comprises detecting the glutathione ethyl ester conjugate by detecting a species resulting from the loss by the glutathione ethyl ester conjugate of a pyroglutamic acid moiety.

12. The method of claim 2 wherein the substrate metabolizing enzyme system comprises a P450 system and the compound comprises glutathione ethyl ester.

13. The method of claim 12 wherein step (a) is conducted with a substrate concentration no greater than 10 μM.

14. The method of claim 2 wherein step (a) is conducted with a substrate concentration no greater than 1 μM.

15. The method of claim 2 wherein step (a) is conducted with a substrate concentration no greater than 0.1 μM.

16. The method of claim 12 wherein step (a) is conducted with a substrate concentration no greater than 0.01 μM.

17. The method of claim 12 wherein the step of detecting at least some of the one or more detectable species comprises passing the detectable species into a mass spectrometer.

18. The method of claim 17 wherein the mass spectrometer comprises a tandem mass spectrometer.

19. The method of claim 17 or 18 wherein the mass spectrometer comprises a triple quadropole mass spectrometer.

20. The method of claim 19 wherein the one or more detectable species are subjected to electrospray ionization prior to passing them into the mass spectrometer.

21. The method of claim 20 wherein the one or more detectable species are subjected to high performance liquid chromatography prior to subjecting them to electrospray ionization.

22. The method of claim 21 wherein the one or more detectable species are passed through a microbore capillary for the electrospray ionization.

23. The method of claim 17 or 18 wherein the one or more detectable species are subjected to high performance liquid chromatography prior to passing them into the mass spectrometer.

24. The method of claim 23 wherein the one or more detectable species are subjected to solid phase extraction prior to passing them into the mass spectrometer.

25. The method of claim 2 wherein the substrate is a drug.

26. The method of claim 25 wherein the reactive species are reactive metabolites.

27. An in vitro method of assessing the possible toxicity of drugs in vivo comprising carrying out the method of any of claims 25 to 26 on a drug and assessing the drug as possibly being toxic in vivo if the level or one or more detectable species or of one or more reactive metabolites or of both is or are above one or more predetermined levels.

28. The method of claim 2 wherein the substrate is a potential drug candidate.

29. The method of claim 28 wherein the reactive species are reactive metabolites.

30. A sensitive and selective in vitro method of assessing the possible toxicity of potential drug candidates in vivo comprising carrying out the process of any of claims 28 to 29 on a potential drug candidate and assessing the potential drug candidate as possibly being toxic in vivo if the level of one or more of the detectable species or of one or more of the reactive metabolites is or are above one or more predetermined levels.

31. A sensitive and selective in vitro method of assessing the possible toxicity of potential drug candidates in vivo comprising:
(a) providing a plurality of potential drug candidates;
(b) carrying out the process of any of claims 28 to 29 on one of the potential drug candidates;
(c) repeating step (b) with a different one of the potential drug candidates; and
(d) assessing each of the potential drug candidates for which the process of any of claims 28 to 29 is carried out as possibly being toxic in vivo if the respective level of one or more detectable species or of one or more reactive metabolites is or are above one or more predetermined levels.
